(19) United States
(12) Patent Application Publication
(10) Pub. No.: US 2018/0200230 A1
(43) Pub. Date: Jul. 19, 2018

(54) METHODS OF TREATING NEURODEGENERATIVE DISEASES USING INDANE ACETIC ACID DERIVATIVES WHICH PENETRATE THE BLOOD BRAIN BARRIER

(71) Applicant: DARA BioSciences, Inc., Raleigh, NC (US)

(72) Inventor: Mary Katherine Delmedico, Raleigh, NC (US)

(73) Assignee: DARA BioSciences, Inc., Raleigh, NC (US)

(21) Appl. No.: 15/863,498

(22) Filed: Apr. 2, 2018

Related U.S. Application Data
(63) Continuation-in-part of application No. 14/477,114, filed on Sep. 4, 2014, now abandoned.
Application No. 14/477,114 is a continuation-in-part of application No. 14/013,801, filed on Aug. 29, 2013, now abandoned.
Application No. 14/013,801 is a continuation-in-part of application No. 13/375,878, filed on Feb. 1, 2012, now abandoned.
(60) Provisional application No. 61/184,157, filed on Jun. 4, 2009.

(51) Int. Cl. A61K 31/421; A61P 25/28; A61P 25/14; A61P 25/16; A61K 45/06

(52) U.S. Cl. CPC A61K 31/421; A61P 25/28; A61K 45/06; A61P 25/16; A61P 25/14

(57) ABSTRACT

This invention describes the use of indane acetic acid derivatives which are dual PPAR delta/gamma agonists, and which penetrate the Blood Brain Barrier and achieve effective brain to plasma drug levels at non-toxic doses, for the treatment of neurodegenerative diseases including one or more of the following: Alzheimer's Disease (AD); Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS).

This application is a Continuation-In-Part of U.S. application Ser. No. 14/477,114 filed on Sep. 4, 2014, which is a Continuation-In-Part of Ser. No. 14/013,801 filed on Aug. 29, 2013, which is a Continuation-In-Part of U.S. application Ser. No. 13/375,878 filed on Feb. 1, 2012, which is a 371 of PCT application number PCT/US2010/037227 filed on Jun. 3, 2010, which claims priority of U.S. provisional application No. 61/184,157 filed on Jun. 4, 2009, which are incorporated herein in their entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### A. Field of the Invention

The present invention relates to the use of brain penetrating indane acetic acids and their derivatives, which are dual PPAR delta and gamma agonists, for the treatment of neurodegenerative diseases such as AD (Alzheimer's Disease), ALS (Amyotrophic Lateral Sclerosis), MS (Multiple Sclerosis), FTD (Frontal Temporal Dementia), HD (Huntington's Disease), PD (Parkinson's Disease), Progressive Supranuclear Palsy (PSP), Dementia with Lewy Bodies (DLB), and CBD (Corticobasal Degeneration).

### B. Description of Related Art

Indane acetic acids PPAR agonists detailed here in were disclosed in Lowe et al US 2005/0075338 A1 Apr. 7, 2005, which was Continuation of application Ser. No. 10/205,839, filed on Jul. 25, 2002, now (U.S. Pat. No. 6,828,335 B2; 2004). The Lowe et al patent (U.S. Pat. No. 6,828,335 B2; 2004) is a composition of matter patent. This instant patent is a method of use patent for the compounds of the indane acetic acids described in U.S. Pat. No. 6,828,335 B2; 2004.

PPARs (peroxisome proliferator-activated receptors, are a family of ligand-activated transcription factors that play an essential role in cellular processes such as cell differentiation, inflammation, and metabolism. The PPARs are ligand-activated transcription factors belonging to the nuclear hormone receptor superfamily, and exist as three different isoforms: PPARα, PPARδ (also called β), and PPARγ. PPARs are activated by lipids and fatty acid derivatives, and they carry out essential functions in lipid homeostasis, glucose metabolism, energy production and cellular differentiation. PPARs are expressed in a variety of CNS related cell types including microglia, astrocytes, oligodendrocytes and neurons. PPAR activation can modulate the immune response, stimulate metabolic and mitochondrial function, promote axon growth, and induce formation of myelinating oligodendrocytes among other disease modifying affects. Compounds which have individual, or single, PPARα, PPARδ, or PPARγ agonist activity are thought to have some potential as systemic therapeutics. Dual or triple PPAR isoform agonists are not well studied and their potential as therapeutics is not well understood. Only recently have there been discovered compositions with dual PPARδ and PPARγ agonist activity where PPARδ activity is greater than PPARγ and PPARα activity.

For a therapeutic to be effective in treating a neurodegenerative disease, it must be able pass from blood through the blood-brain barrier (BBB) and into the brain extracellular fluid (BECF) in the central nervous system (CNS). The blood-brain barrier is formed by endothelial cells, which are connected by tight cell junctions. The blood-brain barrier allows the passage of water, gases, and lipid-soluble molecules by passive diffusion, as well as the selective transport of molecules such as glucose and amino acids that are crucial to neural function. The blood-brain barrier also eliminates lipophilic molecules by way of an active transport mechanism mediated by P-glycoprotein (P-gp), or other efflux transporters such as Organic anion transporter 3 (Oat3) and the peptide transporter 2 (PEPT2). For a neurodegenerative therapeutic to be effective, it has to achieve balance between passive diffusion in through the BBB, and active elimination out of the brain by the P-gp transporter, or other transporters. P-gp is an ATP-dependent, drug efflux pump for xenobiotic compounds with broad tissue distribution including the endothelia cells of the BBB. One measure of whether a small molecule penetrates the BBB and is not rapidly transported out, is the brain to plasma ratio of the drug. This is measured in pre-clinical animal models by determining plasma concentration vs time curves as in a standard pharmacokinetic study, and in addition harvesting brains and determining whole brain concentrations over time. The brain to plasma concentration ratio can them be determined at any time point, such as the Cₘₐₓ, or for the entire time curve (AUC, area under the curve). Alternatively, brain exposure can be determined by measuring drug concentrations in ventricular and lumbar CSF. Clinically, brain FDG-PET can be used as an indirect measure of the pharmacological effect of a therapeutic and of brain levels of drug.

Treating a neurodegenerative disease such as Alzheimer's disease is fundamentally different than treating a systemic disease such as Type 2 diabetes. Lowe et al (U.S. Pat. No. 6,828,335 B2; 2004) teaches administration of indane acetic acids to subjects suffering from Type II diabetes mellitus, hyperchlolesterolemia, hypertriglyceridemia, etc. However, these diseases and other such systemic diseases are distinct from neurodegenerative diseases, and it would be surprising if a systemic agent because the brain is a separate organ uniquely isolated by the tight epithelial cell junctions of the BBB. Because of the control of the Blood Brain Barrier, systemic measures of disease such as serum triglycerides do not translate to brain diseases. The Bisgaier et al (WO 00/38498 A1; 1999) teaches that administering an Alzheimer's disease “preventing or treating” or “alleviating” amount of a plasma-triglyceride lowering agent, and that the plasma-triglyceride level lowering agent is selected from the group consisting of fibrates (clofibrate, gemfibrozil, fenofibrate, etc), niacin, thazolinediones, niacin, EPA and so forth. Bisgaier et all does not teach administration of indane acetic acids, but much more importantly it teaches plasma triglyceride lowering as a measure for neurodegenerative disorders, which one skilled in the art would know are not relevant to neurodegenerative disorders. Exercise is and was known to lower serum triglycerides, but exercise does not cure Alzheimer's disease.

Perhaps most important is that Bisgaier is concerned with plasma concentrations of triglycerides, and does not begin to consider the need of an agent directed towards neurological diseases to get into the brain and have an effect on brain chemistry. Plasma concentrations of triglyceride, the basis of the Bisgaier patent are by definition plasma concentrations and not brain concentrations, but Alzheimers is a brain disease.

Secondly, if in fact lowering levels of plasma concentrations of triglycerides, were in some way related to “treatment, prevention or alleviating” Alzheimers Disease (AD), then triglycerides would be in fact a biomarker for AD, with lower plasma triglyceride levels correlating to less AD, and higher plasma concentrations would relate to worse AD. One skilled in the art of AD knows that this is not the case now, was not the case in 2009, and in fact triglycerides have never been correlated to severity of AD. In fact, in Bisgaier et al, in Table 1 of Example 1, there is a table which compares TG levels in control and AD patients. The control subjects and AD subjects have TG levels that are not statistically significantly different (p=0.249). A p value of less than 0.05 is considered statistically significant in a Student T-test. Clinical trials have failed by not reaching this level of significance, so it is not a trivial difference. Consider some other aspects of measuring Triglycerides (TGs) in plasma. First this is a very common measurement, every time you have clinical chemistries done, TGs get measured, with the primary intent of finding out if you have a serious liver malfunction. So, there is an incredible amount of data out there on TG levels in normal patients, mild cognitive impairment (MCI) and AD subjects, and subjects with all sorts of neurological disorders. TGs are not biomarkers for AD, otherwise it would have been very well known, as so much plasma TG data has been collected over the years. Secondly, the actual plasma TG measurement, not only measures triglycerides, but also diglycerides and monoglycerides. Not only that, but the TG plasma test in no way distinguishes between different types of triglycerides. A triglyceride is a glycerol with three free fatty acids hooked onto it. Some triglycerides are known to be good for health, such as those from fish and vegetables, where as some are detrimental to health. There are short chain, medium chain, long chain, unsaturated, saturated, and branched, triglycerides. The clinical chemistry TG test does not distinguish among any of these, it just measures the total. To one skilled in the art of metabolic diseases and AD, a simple plasma test for TGs is a very crude, unsophisticated tool, not capable of teasing out the complex biology of AD. The role of TGs in AD is complicated, and it is clear to one skilled in the art, that the crude, plasma, test for TGs is no match for this difficult disease.

There are many, many biological mechanisms by which TG levels can be raised or lowered. Simply fasting can affect levels by as much as ten-fold, and pharmaceutical agents can affect plasma TG levels in many different ways. New drugs are monitored for whether they lower plasma TGs mostly as a measure of whether they cause liver toxicity. Lowering Aβ levels requires a whole different set of biological mechanisms that are not related to lowering plasma TG levels and the prior art certainly does not supply them.

A 2009 paper that studied a number of parameters including existence of Metabolic syndrome, High blood pressure, High waist circumference, High plasma triglycerides, Low LDL cholesterol, and high glycemia in the ˜7,800 subject French Three City study, published in Diabetes Care 2009, is the a definitive study on TG and AD. In the Diabetes Care 2009 paper, the association between high plasma triglycerides and Alzheimer's Disease is reported to have a Hazard ratio of 0.9 (0.57 to 1.43) with a p value of 0.67 (not significant) when consider as a separate risk factor for AD, and a Hazard ratio of 1.00 (0.61-1.64) with a p value of 0.998 (very non-statistically significant) when calculated in combination with the other risk factors. When a hazard rate is one, it means the folks with high TGs are getting AD at the same rate as the folks with low TGs. Not only are these hazard rates not statistically significant, but they are both 1 or close to one. Thus, in these 7077 patients, over 4 years there was no statistically significant link between high plasma TGs and AD. This is the state of the art and makes the point clearly that one skilled in the art would not consider plasma TGs to be of any significance in Alzheimer's disease.

Plasma triglycerides are not only scientifically irrelevant to AD, but as reported in the Diabetes Care 2009 publication, are not even coincidentally connected to AD. Thus, the current state of affairs in 2009, and now, did not, and does not present even a “reasonable chance” that lowering TG levels in general would treat prevent, alleviate, or do anything for AD.

The brain has many different cell types. PPARs are known to be involved in the biology of the different brain cells.

Microglia are the primary immune effector cells of the CNS. Neurodegenerative diseases often involve inflammation. Activated microglia release inflammatory cytokines that further recruit circulatory monocytes to the injured brain tissue. PPARγ and PPARδ are critical transcriptional modulators that influence microglia phenotype. PPAR activation of microglia promotes phagocytosis of protein aggregates and is neuroprotective. PPAR induced microglia might be beneficial by promoting removal of cellular debris such as amyloid beta oligomers or plaques.

Astrocytes are the most numerous and diverse non-neuronal support cells (neuroglial) in the CNS, and display a remarkable heterogeneity in their morphology and function. They have numerous projections that anchor neurons to their blood supply. Astrocytes can release damaging inflammatory molecules that cause neuron loss. Modulating inflammation is one of the best-studied roles of PPAR activation in astrocytes and has been examined in multiple experimental models. The responsiveness of astrocytes to PPAR agonists, positions astrocytes in an important role in treatment of neurodegenerative disease.

Oligodendrocytes are myelinating cells of the CNS, which are vulnerable to both inflammatory cytokines and chemokines, and to oxidative damage from reactive oxygen species (ROI). Loss of myelinating oligodendrocytes exposes axons to cytokines and ROIs, leading to axon degeneration. Since myelin is composed of lipid, and PPARs play a major role in lipid metabolism, PPARs may help regulate the function of oligodendrocytes. Targeting oligodendrocytes through PPAR agonists may enhance production and maturation of oligodendrocyte precursor cells (OPCs) and repopulate lost oligodendrocytes and maintain myelination and the integrity of axons.

Neuron loss is a devastating and permanent effect of neurodegeneration. Mitochondrial dysfunction correlates with neuronal cell death, functional impairment, and cognitive deficit since energy production by mitochondria is essential for survival of all cells including neurons. PPAR agonists have been extensively studied for their role in modulating metabolism and energy production. Activation of PPAR receptors by fatty acids promotes mitochondrial β-oxidation increasing cellular energy production. PPAR agonists may induce a clearance mechanism for the amyloid-beta.

There are a number of examples of neurodegenerative diseases that are not currently well serviced by therapeutics. Some of these diseases are described below.

Alzheimers Disease (AD) a chronic, progressive, neurodegenerative disease that is the cause of 60% to 70% of cases of dementia. The most common early symptom is short-term memory loss, and as the disease advances, symptoms can include language difficulties, disorientation, mood swings, loss of motivation, poor self care, and behavior issues. Over time, bodily functions are lost, ultimately leading to death. The average life expectancy following diagnosis is three to nine years. The cause of Alzheimer's disease is not well understood. About 70% of the risk is believed to be genetic. Type 2 Diabetes is also a significant risk factor, along with a history of head injuries, depression, or hypertension. The disease process is associated with amyloid plaques and tau neurofiburilary tangles in the brain. Diagnosis is based on a history of the illness and cognitive testing with medical imaging. Postmortem examination of brain tissue is needed for a definite diagnosis. Alzheimer's disease is characterised by loss of neurons and synapses in the cerebral cortex and certain subcortical regions. This loss results in gross atrophy of the affected regions, including degeneration in the temporal lobe and parietal lobe, and parts of the frontal cortex and cingulate gyrus. Studies using MRI and PET have documented reductions in the size of specific brain regions in people with AD as they progressed from mild cognitive impairment to Alzheimer's disease. Many different brain cell types such as astrocytes, microglia, oligodendrocytes and white matter and gray matter neurons are thought to play a role in AD neurodegeneration. There are approximately 30 million people worldwide with AD, mostly in people over 65 years of age, and leads to about 2 million deaths per year. There are no medications to stop or reverse the progression of AD.

Huntington's disease (HD) is a genetic neurodegenerative disorder that affects muscle coordination and leads to mental decline and behavioral symptoms. The disease results from mutations in the Huntington protein. Initial symptoms include problems with mood or cognition, lack of coordination and unsteady. Physical abilities gradually worsen until coordinated movement becomes difficult and mental abilities generally decline into dementia. HD causes an abnormal increase in astrocytes, and activation of microglia cells. The occurrence of HD is 50-100 cases per million people, with geographic variability due to ethnicity, and past immigration patterns. HD affects the whole brain, but effects are seen in the basal ganglia, which is composed of the caudate nucleus and putamen, the substantia nigra, layers of the cerebral cortex, the hippocampus, purkinje cells in the cerebellum, lateral tuberal nuclei of the hypothalamus and parts of the thalamus.

Parkinson's disease (PD) is the second most common neurodegenerative disorder of the CNS after Alzheimer's Disease (AD). PD affects approximately 1% of the population over the age of 65 and 4-5% of the population over 65. It is a progressive complex, chronic and debilitating disease, which initially produces motor dysfunction but ultimately affects the mind and personality in a large percentage of patients. The pathological hallmark of PD is degeneration of neuromelanin-containing dopaminergic neurons of the substantia nigra (SN), which leads to dopamine (DA) deficiency in the striatum. Another pathological hallmark of the disease is the presence of intraneuronal proteinaceous cytoplasmic inclusions, termed Lewy Bodies. These protein aggregates are composed of numerous proteins including a-synuclein, ubiquitin, and neurofilaments, and are found in all affected brain regions. The clinical features of PD include tremor, bradykinesia, rigidity and impaired balance. Multiple molecular pathways including oxidative stress, mitochondrial dysfunction, inflammation and apoptosis are involved in pathophysiology of PD. PD is the progressive loss of dopaminergic neurons in substantia nigra pars compacta. In PD the putamen plays a key role because its inputs and outputs are interconnected to the substantia nigra and the globus pallidus.

Amyotrophic lateral sclerosis (ALS), also known as motor neuron disease (MND) and Lou Gehrig's disease, involves the death of motor neurons that control voluntary muscles. The disease usually starts around the age of 60 but earlier for inherited cases. The average survival is three to four years, with most patients dying from respiratory failure. In Europe and the United States, the disease affects about 20 people per million people per year. ALS affects about 30,000 Americans. Amyotrophic comes from Greek and translates to “no muscle nourishment”. Common ALS symptoms include stiff muscles, muscle twitching, and gradually muscle wasting and weakening, leading to difficulty speaking, swallowing, and eventually breathing. The defining feature of ALS is the death of upper and lower motor neurons in the motor cortex of the brain, the brain stem, and the spinal cord. Motor neurons develop protein-rich inclusions in their cell bodies and axons, possibly due to defects in protein degradation. Mitochondrial problems have been linked to ALS. In spinal motor neurons, misfolded mutant SOD1 protein preferentially binds to mitochondrial membranes, and accumulation of SOD1 mutations in mitochondria leads to mitochondrial dysfunction, oxidative stress and subsequent defects in neuronal physiology.

Frontotemporal Dementia (FTD) is the most common neurodegenerative disease in people under the age of 60. FTD accounts for 20% of young-onset dementia cases, equally affecting men and women. It often occurs in late middle age and is distinct from AD and psychiatric disorders. Individuals with FTD display personality, behavior, or language problems associated with atrophy of the frontal and temporal brain lobes, but memory is typically spared. Approximately 40% of cases have family histories with dominantly inherited genetic mutations that cause FTD. Progranulin deficiency is thought to cause frontotemporal dementia. Neuropathological signs of progranulin-deficient FTD include dystrophic neurites, inflammation, microglial activation, and severe neuron loss in the frontal and temporal lobes. Progranulin is linked to obesity and insulin resistance. Pathologic changes in FTD are observed in the striatum, thalamus, substantia nigra, and hippocampus.

Corticobasal degeneration (CBD) or corticobasal ganglionic degeneration (CBGD) is a rare, progressive neurodegenerative disease involving the cerebral cortex and the basal ganglia with typical onset in the sixth to eighth decades of life. Typical mean survival is 7 years from symptom onset. Core clinical features are progressive asymmetric rigidity and apraxia, accompanied by symptoms and signs of cortical (motor, sensory or association cortices) and extrapyramidal dysfunction. Cortical signs include cortical sensory loss, alien limb phenomena, myoclonus, apraxia, pyramidal motor signs, agrammatic aphasia, apraxia of speech and visuospatial impairment. Extrapyramidal involvement includes dystonia and levodopa nonresponsive Parkinsonism (rigidity, tremor and bradykinesia). The characteristic histopathological findings are neuronal loss and numerous swollen achromatic neurons. These features are seen throughout the brain, but certain regions are more severely affected including: frontoparietal cortex, subcortical structures, striatum, and substantia nigra. Clinical findings may be explained by the distribution of cortical damage: cortical sensory loss and apraxia occur with parietal lesions; frontal or parietal lesions can produce alien limb syndrome, mirror movements, and apraxia; and posterior inferior frontal lesions can produce agrammatic aphasia. Severe rigidity, dystonia and tremor have been reported and are thought to reflect damage to the sensorimotor cortex. CDB is often confused with progressive supranuclear palsy (PSP). Both PSP and CBD result in similar symptoms, and both display tauopathies upon histological inspection.

Individuals diagnosed with Parkinson's Disease often exhibit similar movement dysfunction as those diagnosed with CBD. Some other neurodegenerative diseases including Alzheimer's disease (AD), dementia with Lewy bodies (DLB), and frontotemporal dementia (FTD) also show commonalties with CBD.

Progressive supranuclear palsy (PSP; or Steele-Richardson-Olszewski syndrome) is a neurodegenerative disease involving the gradual deterioration and death of specific volumes of the brain. It is considered a tauopathy. Approximately six people per 100,000 population have PSP, males and females are affected equally, and there is no racial, geographical or occupational correlation. Initial symptoms in most cases are loss of balance, lunging, fast walking, bumping into objects, personality changes, visual symptoms, and falls. Later symptoms are dementia, loss of inhibition and ability to organize information, slurring of speech, difficulty swallowing, and difficulty moving the eyes. The cause of PSP is unknown. Fewer than 1% of those with PSP have a family connection. A variant in the gene for tau protein called the H1 haplotype, located on chromosome 17, has been linked to PSP. About two-thirds of the general population have this mutation. Therefore, the H1 haplotype appears to be necessary but not sufficient to cause PSP. The affected brain cells in PSP include both neurons and glial cells. The neurons display neurofibrillary tangles. The principal areas of the brain affected are the: basal ganglia, particularly the subthalamic nucleus, substantia nigra and globus pallidus; brainstem, cerebral cortex, particularly that of the frontal lobes; dentate nucleus of the cerebellum; spinal cord, particularly the area where some control of the bladder and bowel resides. Some consider PSP, corticobasal degeneration, and frontotemporal dementia to be variations of the same disease. PSP has been shown to co-exist with Pick's disease.

Dementia with Lewy bodies (DLB) is a progressive dementia with symptoms that may include changes in alertness, excessive sleep movement, mood changes, visual hallucinations, slowness of movement, trouble walking, and rigidity. DLB is less common Alzheimer's disease and vascular dementia, typically beginning after the age of 50, and effecting males more than females. About 0.1% of those over 65 are affected. Life expectancy following diagnosis is roughly eight years. Most cases of DLB appear sporadically and it is not thought to have a strong hereditary link. Similar to AD, the LBD risk is heightened with inheritance of the ε4 allele of the apolipoprotein E (APOE) gene. DLB is characterized by the development of abnormal collections of (alpha-synuclein) protein within the cytoplasm of neurons (known as Lewy bodies). A loss of dopamine-producing neurons in the substantia nigra and putamen, along with a loss of acetylcholine-producing neurons in the basal nucleus of Meynert and elsewhere, is known to occur in those with DLB. Cerebral atrophy also occurs as the cerebral cortex degenerates.

Multiple sclerosis (MS) is a demyelinating disease in which the protective myelin covers of nerve cells in the brain and spinal cord are damaged. This damage disrupts the ability of the nervous system to communicate, resulting in multiple symptoms, including physical, and mental problems. MS takes several forms, with new symptoms either occurring in isolated attacks (relapsing forms) or building up over time (progressive forms). The three main characteristics of MS are the formation of lesions in the central nervous system (plaques), inflammation, and the destruction of myelin sheaths of neurons. These lesions most commonly affect the white matter in the optic nerve, brain stem, basal ganglia, and spinal cord, or white matter tracts close to the lateral ventricles. Physiologically, MS involves the loss of oligodendrocytes, the cells responsible for creating and maintaining a fatty layer known as the myelin sheath. Without myelin neurons cannot effectively conduct electrical signals. Re-myelination can occur, but the diseased oligodendrocytes are unable to keep up with the damage. In addition, increases in astrocyte cell numbers and the resulting destruction of proximal neurons is also responsible for creation of lesions. The possible causes for MS include genetics and environmental factors such as infections. Multiple sclerosis is the most common autoimmune disorder affecting the central nervous system. Several million people are affected globally with rates varying in different regions of the world and among different populations. The disease usually begins between the ages of 20 and 50 and is twice as common in women as in men.

### SUMMARY OF THE INVENTION

The present invention comprises a method of treating a subject having a neurodegenerative disease comprising administering an indane acetic acid, dual Peroxisome Proliferator-Activated Receptor (PPAR) delta and gamma agonist, which penetrates the blood brain barrier (BBB) and achieves pharmacologically useful concentrations in the brain, wherein the neurodegenerative disease is selected from the group comprising:
a) Alzheimer's Disease (AD);
b) Huntington's Disease (HD);
c) Parkinson's Disease (PD);
d) Amyotrophic Lateral Sclerosis (ALS);
e) Frontal Temporal Dementia (FTD);
f) Corticobasal Degeneration (CBD);
g) Progressive Supranuclear Palsey (PSP);
h) Dementia with Lewy Bodies (DLB);
i) Multiple Sclerosis (MS);
and wherein, the indane acetic acid, comprises a compound of Formula I, or a pharmaceutically acceptable salt, ester prodrug, stereoisomer, enantiomer, racemate or a combination thereof wherein Formula I is:

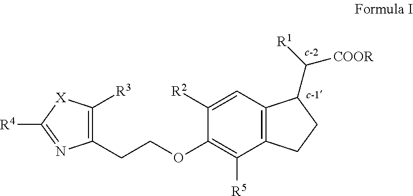

wherein
R is H, Na⁺, Li⁺, Ca⁺, K⁺, N⁺(C1-C6)₄, or C1-C6 alkyl;
R¹ is H, COOR, C₃-C₈ cycloalkyl, or C₁-C₆ alkyl, C₂-C₆ alkenyl, or C₁-C₆ alkoxy, each of which may be unsubstituted or substituted with fluoro, methylenedioxyphenyl, or phenyl which may be unsubstituted or substituted with R⁶, “c-2” is defined as the second carbon of the acetic acid portion of Formula I, and “c-1′” is the first carbon of the indane group of Formula I;
R² is H, halo, or C₁-C₆ alkyl which may be unsubstituted or substituted with C₁-C₆ alkoxy, oxo, fluoro,
or R² is phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, or morpholinyl, each of which may be unsubstituted or substituted with R⁶;
R³ is H, C₁-C₆ alkyl, or phenyl, which may be unsubstituted or substituted with R⁶;
X is O or S;
R⁴ is phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl, each of which may be unsubstituted or singularly or multiply substituted with R⁶, or with phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, morpholinyl, benzodioxolyl, dihydrobenzofuranyl, indolyl, pyrimidinyl or phenoxy, each of which may be unsubstituted or singularly or multiply substituted with R⁶;
or R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶, each of which may be substituted with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl, each of which may be unsubstituted or further substituted with R⁶, or any C₁-C₆ alkyl may also be substituted with C₃-C₈ cycloalkyl or with phenoxy which may be unsubstituted or substituted with R⁶ or with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl, each of which may be unsubstituted or substituted with R⁶, or
R⁵ is H, halo or C₁-C₆ alkyl optionally substituted with oxo; and
R⁶ is halo, CF₃, C₁-C₆ alkyl optionally substituted with oxo or hydroxy, or C₁-C₆ alkoxy optionally substituted with fluoro; and wherein R³ may be attached to the heterocyclic moiety of the compound of Formula I at either the 4 or 5 position, and, accordingly, the remaining portion of the molecule will be attached at the remaining available carbon atom.

In other embodiments the dual PPAR delta and gamma agonist, which penetrates the blood brain barrier (BBB), and achieves pharmacologically useful concentrations in the brain, is of Formula I, or is a pharmaceutically acceptable salt, ester prodrug, stereoisomer, enantiomer, racemate or a combination thereof wherein Formula I is:

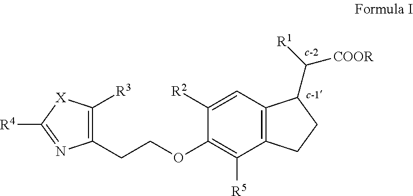

wherein
R is H, Na⁺, Li⁺, Ca⁺, K⁺, N⁺(C1-C6)₄, or C1-C6 alkyl;
R¹ is H, “c-2” is defined as the second carbon of the acetic acid portion of Formula I, and “c-1′” is the first carbon of the indane group of Formula I;
R² is H, halo, or C₁-C₆ alkyl which may be unsubstituted or substituted with C₁-C₆ alkoxy, oxo, fluoro;
R³ is H, C₁-C₆ alkyl, or phenyl, which may be unsubstituted or substituted with R⁶;

### X is O or S;

R⁴ is phenyl, which may be unsubstituted or singularly or multiply substituted with R⁶;
R⁵ is H, halo or C₁-C₆ alkyl optionally substituted with C₁-C₆ alkoxy, oxo, fluoro;
R⁶ is halo, CF₃, C₁-C₆ alkyl optionally substituted with oxo or hydroxy, or C₁-C₆ alkoxy optionally substituted with fluoro; wherein R³ may be attached to the heterocyclic moiety of the compound of Formula I at either the 4 or 5 position and, accordingly, the remaining portion of the molecule will be attached at the remaining available carbon atom.

In another embodiment, the indane acetic acid used to treat a neurodegenerative disease, described above, and which penetrates the blood brain barrier, has rat brain to plasma ratios of greater than 10% 12 hours after oral dosing.

In another embodiment, the indane acetic acid described above is used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS).

In another embodiment, the indane acetic acids used to treat a neurodegenerative disease, which penetrate the blood brain barrier are selected from compounds of Formula I wherein: R is H, Na⁺, Li⁺, Ca⁺, K⁺, N⁺(C1-C6)₄, or C1-C6 alkyl; R¹ is H; R² is H, halo; R³ is H, C₁-C₆ alkyl; X is O or S; R⁴ is phenyl, which may be singularly or multiply substituted with R⁶; R⁵ is H, halo; R⁶ is halo, CF₃, C₁-C₆ alkyl or C₁-C₆ alkoxy; and c-1′ has the S stereochemistry.

In another embodiment, the indane acetic acids described above, which penetrate the blood brain barrier, and are used to treat the listed neurodegenerative diseases, have rat brain to plasma ratios of greater than 10% 12 hours after oral dosing.

In another embodiment, the indane acetic acids described above, which penetrate the blood brain barrier, are used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS).

In another embodiment, the indane acetic acids used to treat a neurodegenerative disease, which penetrate the blood brain barrier are selected from compounds of Formula I wherein: R is H, Na⁺, Li⁺, Ca⁺, K⁺, N⁺(C1-C6)₄, or C1-C6 alkyl; R¹ is H; R² is H, halo; R³ is C₁-C₆ alkyl; X is O; R⁴ is phenyl, which may be singularly or multiply substituted with R⁶; R⁵ is H, halo; R⁶ is halo, CF₃, C₁-C₆ alkyl or C₁-C₆ alkoxy; and c-1′ has the S stereochemistry.

In another embodiment, the indane acetic acids described above which penetrate the blood brain barrier, and are used to treat the listed neurodegenerative diseases, have rat brain to plasma ratios of greater than 10% 12 hours after oral dosing.

In another embodiment, the indane acetic acids described above which penetrate the blood brain barrier, are used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS);

In another embodiment, the indane acetic acids used to treat a neurodegenerative disease, which penetrate the blood brain barrier are selected from compounds of Formula I wherein: R is H, Na⁺, Li⁺, Ca⁺, K⁺, N⁺(C1-C6)₄, or C1-C6 alkyl; R¹ is H, R² is F, R⁵ is F, R³ is C₁-C₆ alkyl, X is O or S, and R⁴ is a phenyl, singularly or multiply substituted with R⁶, wherein R⁶ is halo, CF₃, C₁-C₆ alkoxyl or C₁-C₆ alkyl, and the stereochemistry at c-1′ is defined as S.

In another embodiment, the indane acetic acids described above which penetrate the blood brain barrier, and are used to treat the listed neurodegenerative diseases, have rat brain to plasma ratios of greater than 10% 12 hours after oral dosing.

In another embodiment, the indane acetic acids described above which penetrate the blood brain barrier, are used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS).

In another embodiment, the indane acetic acids used to treat a neurodegenerative disease, which penetrate the blood brain barrier are selected from compounds of Formula I wherein: R is H or Na, R¹ is H, R² is H, R⁵ is H, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S.

In another embodiment, the indane acetic acids described above which penetrate the blood brain barrier, and are used to treat the listed neurodegenerative diseases, have rat brain to plasma ratios of greater than 10% 12 hours after oral dosing.

In another embodiment, the indane acetic acids described above which penetrate the blood brain barrier, are used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS).

In another embodiment, the indane acetic acids used to treat a neurodegenerative disease, which penetrate the blood brain barrier are selected from compounds of Formula I wherein: R is H or Na, R¹ is H, R² is H, R⁵ is H, R³ is C₁-C₆ alkyl, X is S, and R⁴ is a phenyl, singularly or multiply substituted with R⁶, wherein R⁶ is halo, CF₃, C₁-C₆ alkoxyl or C₁-C₆ alkyl, and the stereochemistry at c-1′ is defined as S.

In another embodiment, the indane acetic acids described above which penetrate the blood brain barrier, and are used to treat the listed neurodegenerative diseases, have rat brain to plasma ratios of greater than 10% 12 hours after oral dosing.

In another embodiment, the indane acetic acids described above which penetrate the blood brain barrier, are used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS).

In another embodiment, the indane acetic acids used to treat a neurodegenerative disease, which penetrate the blood brain barrier are selected from the group consisting of the free acid, in the form of a pharmaceutically acceptable salt such as a potassium, sodium, calcium, magnesium, lysine, choline or meglumine salt thereof, and has a structure selected from the group consisting of:

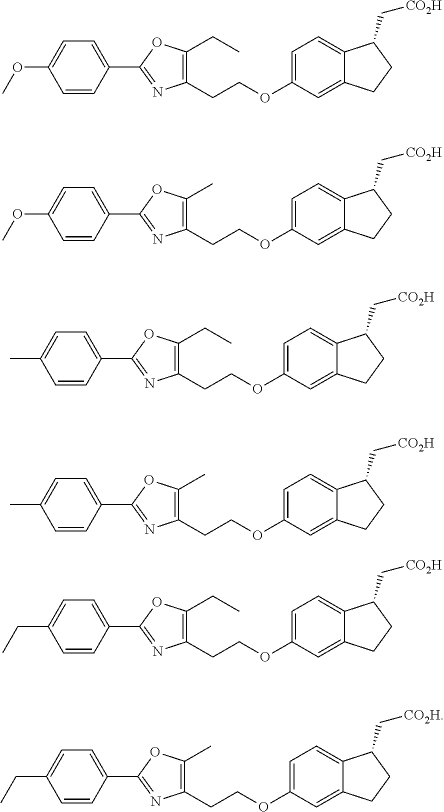 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

In another embodiment, the indane acetic acid used to treat a neurodegenerative disease, is selected from the list of six above and has a rat brain to plasma ratio of greater than 20% 12 hours after oral dosing.

In another embodiment, the indane acetic acid with a rat brain to plasma ratio of greater than 20% 12 hours after oral dosing, is used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS).

In another embodiment, the indane acetic acid, which penetrates the blood brain barrier (BBB), and is used to treat a listed neurodegenerative disease, is a pharmaceutically acceptable salt thereof, wherein the pharmaceutically acceptable salt is selected from the group consisting of ammonium salts, protonated basic amines, and quaternary amines.

In another embodiment, the route of administration for the indane acetic acid which penetrates the blood brain barrier (BBB), and is used to treat one of the listed neurodegenerative diseases, is selected from the group consisting of intravenously, orally, buccally, transdermally, rectally, nasally, optically, intrathecally and intra-cranially.

In another embodiment, the indane acetic acid which penetrates the blood brain barrier (BBB), and is used to treat one of the listed neurodegenerative diseases, is administered with one or more additional therapeutic agents selected from a group consisting of: a therapeutic agent to treat Alzheimer's disease such as Donepezil, Rivastigmine, or Memantine; a therapeutic agent used to treat Parkinson's Disease (PD) such as L DOPA (levodopa), or dopamine agonists; a therapeutic agent used to treat Amyotrophic Lateral Sclerosis (ALS) such as Edaravone (Radicut), or Riluzole; a therapeutic agent used to treat Multiple Sclerosis (MS) such as interferon beta, glatiramer acetate, mitoxantrone, natalizumab, fingolimod, teriflunomide, alemtuzumab, daclizumab, or ocrelizumab; or a therapeutic agent used to treat Huntington's Disease (HD) such as tetrabenazine.

In another embodiment, the indane acetic acid, which penetrates the blood brain barrier and is used to treat the listed neurodegenerative diseases, is: ((1S)-5-{5-ethyl-2-(4-methoxyphenyl)-1, 3-oxazol-4-yl] ethoxy}-2, 3-dihydro-1H-inden-1-yl) acetic acid, sodium salt. (CAS Registry number 1258076-66-2) with the with the structure shown below:

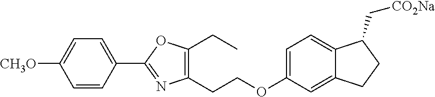

In another embodiment, the indane acetic acid shown above, used to treat a listed neurodegenerative disease, and which penetrates the blood brain barrier, has rat brain to plasma ratios of greater than 20% 12 hours after oral dosing.

In another embodiment, the indane acetic acid shown above, which penetrates the blood brain barrier, and has rat brain to plasma ratios of greater than 20% 12 hours after oral dosing, is used to treat a neurodegenerative disease selected from: Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS);

In another embodiment, the indane acetic acid, which penetrates the blood brain barrier (BBB), is administered intravenously, orally, buccally, transdermally, rectally, nasally, optically, intrathecally, or intra-cranially.

In another embodiment, the indane acetic acid shown above is administered with one or more additional therapeutic agents selected from a group consisting of: a therapeutic agent to treat Alzheimer's disease such as Donepezil, Rivastigmine, or Memantine; a therapeutic agent used to treat Parkinson's Disease (PD) such as L DOPA (levodopa), or dopamine agonists; a therapeutic agent used to treat Amyotrophic Lateral Sclerosis (ALS) such as Edaravone (Radicut), or Riluzole; a therapeutic agent used to treat Multiple Sclerosis (MS) such as interferon beta, glatiramer acetate, mitoxantrone, natalizumab, fingolimod, teriflunomide, alemtuzumab, daclizumab, or ocrelizumab; or a therapeutic agent used to treat Huntington's Disease (HD) such as tetrabenazine.

In another embodiment the indane acetic acid is any such composition which is a duel PPAR delta and gamma agonist and penetrates the blood brain barrier (BBB).

Objects of the present invention will be appreciated by those of ordinary skill in the art from a reading of the referenced patent literature and the Examples and the detailed description of the embodiments, which follow, such description being merely illustrative of the present invention.

### BRIEF DESCRIPTION OF THE DRAWINGS

FIGS. 1-4 show brain scans after various dosages of a compound of the invention.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### A. DEFINITIONS

The terms “about” and “essentially” mean±20 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

References throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these, or their equivalent, in view of the disclosure herein and use of the term “means” is not intended to be limiting.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs. In the event that there is a plurality of definitions for a term used herein, those in this section prevail unless stated otherwise.

As used herein the term “PPAR delta and gamma agonist” and “PPAR delta and gamma activity” refers to agonists where delta activity is greater than gamma activity and gamma activity is greater than alpha activity.

The term “halo” means F, Cl, Br, or I.

The term “C1-C6 alkyl” means a straight or branched saturated hydrocarbon carbon chain of from 1 to about 6 carbon atoms, respectively. Examples of such groups include methyl, ethyl, isopropyl, sec-butyl, 2-methylpentyl, n-hexyl, and the like.

The term “C2-C6 alkenyl” means a straight or branched unsaturated hydrocarbon carbon chain of from 2 to about 6 carbon atoms. Examples of such groups include vinyl, allyl, isopropenyl, 2-butenyl, 3-ethyl-2-butenyl, 4-hexenyl, and the like.

The term “C1-C6 haloalkyl” means a C1-C6 alkyl group substituted by 1 to 3 halogen atoms or fluorine up to the perfluoro level. Examples of such groups include trifluoromethyl, tetrafluoroethyl, 1,2-dichloropropyl, 6-iodohexyl, and the like.

The terms “C3-C6 cycloalkyl” and “C3-C8 cycloalkyl” mean a saturated carbocyclic ring system of from 3 to about 6 carbon atoms or from 3 to about 8 carbon atoms, respectively. Examples of such groups include cyclopropyl, cyclobutyl, cyclopentyl, cyclohexyl, and the like.

The term “C1-C6 acyl” means a C1-C6 alkyl group attached at the carbonyl carbon atom. The radical is attached to the rest of the molecule at the carbonyl bearing carbon atom. Examples of such groups include acetyl, propionyl, n-butanoyl, 2-methylpentanoyl, and the like.

The term “C1-C6 alkoxy” means a linear or branched saturated carbon group having from 1 to about 6 C atoms, said carbon group being attached to an O atom. The O atom is the point of attachment of the alkoxy substituent to the rest of the molecule. Such groups include, but are not limited to, methoxy, ethoxy, n-propoxy, isopropoxy, and the like.

The term “C1-C6 thioalkyl” means a linear or branched saturated carbon group having from 1 to about 6 C atoms, said carbon group being attached to an S atom. The S atom is the point of attachment of the thioalkyl substituent to the rest of the molecule. Such groups include, for example, methylthio, propylthio, hexylthio, and the like.

The term “C1-C6 haloalkoxy” means a C1-C6 alkoxy group further substituted on C with 1 to 3 halogen atoms or fluorine up to the perfluoro level.

The term “C3-C8 cycloalkoxy” means a C3-C8 cycloalkyl group attached to an O atom. The O atom is the point of attachment of the cycloalkoxy group with the rest of the molecule.

The term “phenoxy” means a phenyl group attached to an O atom. The O atom is the point of attachment of the phenoxy group to the rest of the molecule.

The term “6-membered heteroaryl ring” means a 6-membered monocyclic heteroaromatic ring radical containing 1-5 carbon atoms and up to the indicated number of N atoms. Examples of 6-membered heteroaryl rings are pyridyl, pyrimidyl, pyridazinyl, pyrazinyl, triazinyl, and the like.

The term “5- or 6-membered heterocyclic ring” means a 5- or 6-membered ring containing 1-5 C atoms and up to the indicated number of N, O, and S atoms, and may be aromatic, partially saturated, or fully saturated.

The term “c-2” is defined as the second carbon of the acetic acid portion of Formula I, and is used to denote a chiral center when R1 is anything other than H; The term “c-1” is defined as the first carbon of the indane group of Formula I, which is a chiral center in all compounds of Formula I.

The term “optionally substituted” means that, unless indicated otherwise, the moiety so modified may have from one to up to the number of the substituents indicated, provided the resulting substitution is chemically feasible as recognized in the art. Each substituent may replace any H atom on the moiety so modified as long as the replacement is chemically possible and chemically stable. For example, a chemically unstable compound would be one where each of two substituents is bonded to a single C atom through each substituents heteroatom. Another example of a chemically unstable compound would be one where an alkoxy group is bonded to the unsaturated carbon of an alkene to form an enol ether. When there are two or more substituents on any moiety, each substituent is chosen independently of the other substituent so that, accordingly, the substituents can be the same or different.

When the 5- or 6-membered heterocyclic ring is attached to the rest of the molecule as a substituent, it becomes a radical. Examples of 5- or 6-membered heteroaryl ring radicals are furyl, pyrrolyl, thienyl, pyrazolyl, isoxazolyl, imidazolyl, oxazolyl, thiazolyl, isothiazolyl, triazolyl, thiadiazolyl, oxadiazolyl, pyridyl, pyrimidyl, pyridazinyl, pyrazinyl, triazinyl, and the like. Examples of partially unsaturated 5- or 6-membered heterocyclic ring radicals include dihydropyrano, pyrrolinyl, pyrazolinyl, imidazolinyl, dihydrofuryl, and the like. Examples of saturated 5- or 6-membered heterocyclic ring radicals include pyrrolidinyl, tetrahydropyridyl, piperidinyl, morpholinyl, tetrahydrofuryl, tetrahydrothienyl, piperazinyl, and the like. The point of attachment of the radical may be from any available C or N atom of the ring to the rest of the molecule. When the 5- or 6-membered heterocyclic ring is fused to another ring contained in the rest of the molecule, it forms a bicyclic ring. Examples of such 5- and 6-heterocyclic fused rings include pyrrolo, furo, pyrido, piperido, thieno, and the like. The point of fusion is at any available face of the heterocyclic ring and parent molecule.

The term “subject”, as used herein, means a mammalian subject (e.g., dog, cat, horse, cow, sheep, goat, monkey, etc.), and particularly human subjects (including both male and female subjects, and including neonatal, infant, juvenile, adolescent, adult and geriatric subjects, and further including various races and ethnicities including, but not limited to, white, black, Asian, American Indian and Hispanic).

As used herein, “treatment”, “treat”, and “treating” refer to reversing, alleviating, mitigating, or slowing the progression of, or inhibiting the progress of, a disorder or disease as described herein.

As used herein, “prevention”, “prevent”, and “preventing” refer to eliminating or reducing the incidence or onset of a disorder or disease as described herein, as compared to that which would occur in the absence of the measures taken.

As used herein, “an effective amount” refers to an amount that causes relief of symptoms of a disorder or disease as noted through clinical testing and evaluation, patient observation, and/or the like. An “effective amount” can further designate a dose that causes a detectable change in biological or chemical activity. The detectable changes may be detected and/or further quantified by one skilled in the art for the relevant mechanism or process. Moreover, an “effective amount” can designate an amount that maintains a desired physiological state, i.e., reduces or prevents significant decline and/or promotes improvement in the condition of interest. An “effective amount” can further refer to a therapeutically effective amount.

All patents, patent applications and publications referred to herein are incorporated by reference in their entirety. In case of a conflict in terminology, the present specification is controlling.

The difference between the claimed invention and the prior art is very large, such that the claimed invention as a whole, would not have been obvious before the effective filing date of the instant claimed invention.

The instant invention is for the use of indane acetic acid derivatives, which exert their biological activity as PPAR agonists, and penetrate the blood brain barrier (BBB), for treatment of specific neurodegenerative diseases. This novel concept requires that such a compound would penetrate the blood brain barrier and have a pharmacological effect in the brain. That indane acetic acids can achieve significant brain concentrations is surprising, as the general understanding in the literature is that carboxylic acids, which are negatively charged (anionic) at physiological pH values, are poorly brain penetrant. In fact, the ideal structure for blood brain penetration is generally considered to be positively charged (cationic at physiological pH) basic amine. As organic acids, it was also thought likely that indane acetic acids that did get into the brain would be rapidly eliminated from the brain by efflux transporters such as P glycoprotein (Pgp) or organic acid transporters (OATs) effectively limiting their brain to plasma concentrations. However, a study that measured rat brain concentrations of an exemplified compound, and showed that 12 hours after oral dosing, 35% of the amount in the plasma, was found in the brain. This is considered a substantial amount, and certainly enough to have a substantial pharmacological effect. In addition, an in vitro Pgp efflux experiment showed exemplified compounds were not substrates for the human Pgp transporter. Brain penetration was confirmed for an exemplified indane acetic acid in clinical trials by FDG-PET (F18 Fluorodeoxyglucose Positron Emission Tomography).

### B. COMPOUNDS

### (1). Formula I

The present invention encompasses the compounds of Formula I which are PPAR delta and gamma dual agonists,

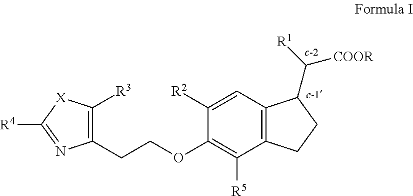

wherein in Formula I

R is H or C₁-C₆ alkyl;

R¹ is H, COOR, C₃-C₈ cycloalkyl, or C₁-C₆ alkyl, C₂-C₆ alkenyl, or C₁-C₆ alkoxy each of which may be unsubstituted or substituted with fluoro, methylenedioxyphenyl, or phenyl which may be unsubstituted or substituted with R⁶;

R² is H, halo, or C₁-C₆ alkyl which may be unsubstituted or substituted with C₁-C₆ alkoxy, oxo, fluoro, or

R² is phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, or morpholinyl,
each of which may be unsubstituted or substituted with R⁶;

R³ is H, C₁-C₆ alkyl, or phenyl, which may be unsubstituted or substituted with R⁶;

X is O or S;

R⁴ is phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,

each of which may be unsubstituted or singularly or multiply substituted with R⁶, or with phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, morpholinyl, benzodioxolyl, dihydrobenzofuranyl, indolyl, pyrimidinyl or phenoxy,

each of which may be unsubstituted or singularly or multiply substituted with R⁶;

R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶,
each of which may be substituted with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
   each of which may be unsubstituted or further substituted with R⁶,
or
C₁-C₆ alkyl may also be substituted with C₃-C₈ cycloalkyl or with phenoxy which may be unsubstituted or substituted with R⁶ or with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl,
   each of which may be unsubstituted or substituted with R⁶, or

R⁵ is H, halo or C₁-C₆ alkyl optionally substituted with oxo; and

R⁶ is halo, CF₃, C₁-C₆ alkyl optionally substituted with oxo or hydroxy, or C₁-C₆ alkoxy optionally substituted with fluoro; or a pharmaceutically acceptable salt, ester prodrug, stereoisomer, diastereomer, enantiomer, racemate or a combination thereof.

R³ may be attached to the heterocyclic moiety of the compound of Formula I at either the 4 or 5 position (i.e., at either available carbon atom) and, accordingly, the remaining portion of the molecule will be attached at the remaining available carbon atom.

In some embodiments, the compound of Formula I has structure as described above and R is potassium, sodium, calcium, magnesium, lysine, choline or meglumine salt thereof.

In other embodiments, for the compound of Formula I, R is H, R¹ is H, R² is H, R⁵ is H, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, or a pharmaceutically acceptable salt thereof.

In other embodiments, for the compound of Formula I, R is H, R¹ is H, R² is H, R⁵ is H, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S, or a pharmaceutically acceptable salt thereof.

In other embodiments, for the compound of Formula I, R is H, R¹ is H, R² is H, R⁵ is H, R³ is C₁-C₆ alkyl, X is S, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S, or a pharmaceutically acceptable salt thereof.

In other embodiments, for the compound of Formula I, R is H, R¹ is H, R² is F, R⁵ is H, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S, or a pharmaceutically acceptable salt thereof.

In other embodiments, for the compound of Formula I, R is H, R¹ is H, R² is H, R⁵ is F, or R² and R⁵ are F, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S, or a pharmaceutically acceptable salt thereof.

In other embodiments, for the compound of Formula I, R is H, R¹ is H, R² is H, R⁵ is H, R³ is C₁-C₆ alkyl, X is O, and R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as R, or a pharmaceutically acceptable salt thereof.

In one embodiment, the compound of Formula I is either the free acid or the potassium, sodium, calcium, magnesium, lysine, choline or meglumine salt of one of the following structures:

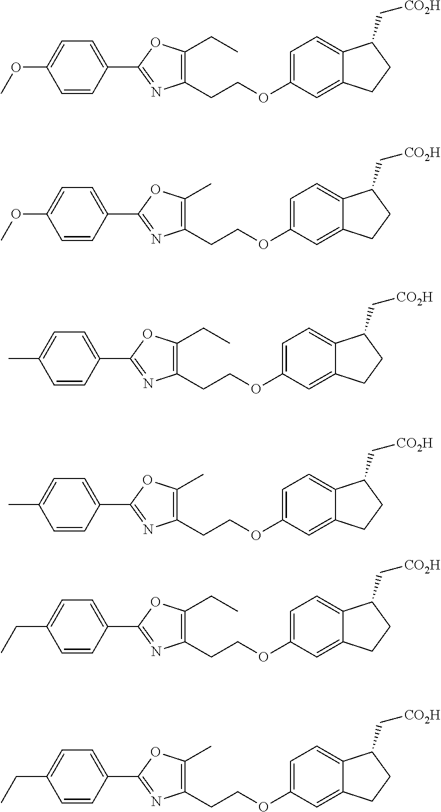 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

In another embodiment, the compound of Formula I is a potassium or sodium salt of the structures:

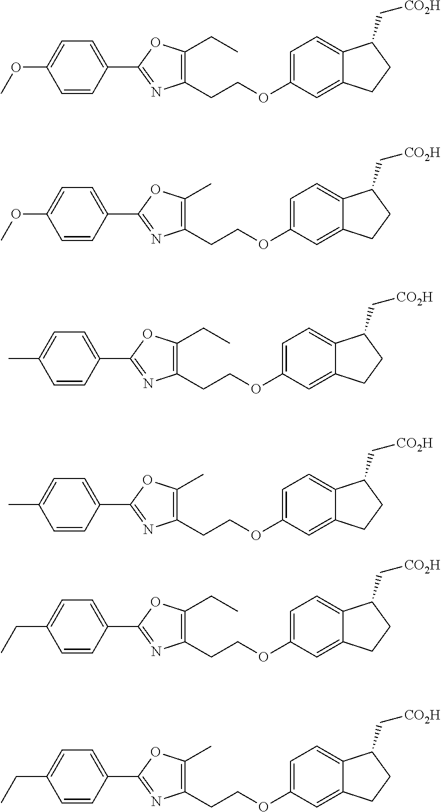 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

Exemplary compounds of Formula I are listed in Table 1 as the free acid, but may also be a pharmaceutically acceptable salt thereof.

TABLE 1
Illustrative Examples of Compounds of Formula I

| | | | | | Formula I | |
|---|---|---|---|---|---|---|
| 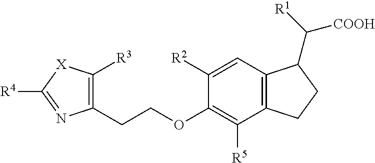 | | | | | | |
| Entry | | | | | | |
| No. | R¹ | R² | R³ | R⁴ | R⁵ | X |
| 1 | H | H | Me | Ph | H | O |
| 2 | H | H | Me | 2-F-Ph | H | O |
| 3 | H | H | Me | 2-Cl Ph | H | O |
| 4 | H | H | Me | 2-Me Ph | H | O |
| 5 | H | H | Me | 3-F-Ph | H | O |
| 6 | H | H | Me | 3-Cl Ph | H | O |
| 7 | H | H | Me | 3-CF₃ Ph | H | O |
| 8 | H | H | Me | 3-Me Ph | H | O |
| 9 | H | H | Me | 3-MeO Ph | H | O |
| 10 | H | H | Me | 4-F-Ph | H | O |
| 11 | H | H | Me | 4-Cl Ph | H | O |
| 12 | H | H | Me | 4-CF₃ Ph | H | O |
| 13 | H | H | Me | 4-Me Ph | H | O |
| 14 | H | H | Me | 4-Et Ph | H | O |
| 15 | H | H | Me | 4-MeO Ph | H | O |
| 16 | H | H | Me | 4-EtO Ph | H | O |
| 17 | H | H | Me | 2,3-di-F Ph | H | O |
| 18 | H | H | Me | 2,4-di-F Ph | H | O |
| 19 | H | H | Me | 3,4-di-F Ph | H | O |
| 20 | H | H | Me | 2,6-di-F Ph | H | O |
| 21 | H | H | Me | 2,3-di-Cl Ph | H | O |
| 22 | H | H | Me | 3,4-di-Cl Ph | H | O |
| 23 | H | H | Me | 2,4-di-Cl Ph | H | O |
| 24 | H | H | Me | 2,6-di-Cl Ph | H | O |
| 25 | H | H | Me | 2,3-di-Me Ph | H | O |
| 26 | H | H | Me | 2,4-di-Me Ph | H | O |
| 27 | H | H | Me | 3,4-di-Me Ph | H | O |
| 28 | H | H | Me | 2,6-di-Me Ph | H | O |
| 29 | H | H | Me | 2,3-di-MeO Ph | H | O |
| 30 | H | H | Me | 2,4-di-MeO Ph | H | O |
| 31 | H | H | Me | 3,4-di-MeO Ph | H | O |
| 32 | H | H | Et | Ph | H | O |
| 33 | H | H | Et | 2-Cl Ph | H | O |
| 34 | H | H | Et | 2-Me Ph | H | O |
| 35 | H | H | Et | 3-F-Ph | H | O |
| 36 | H | H | Et | 3-Cl Ph | H | O |
| 37 | H | H | Et | 3-CF₃ Ph | H | O |
| 38 | H | H | Et | 3-Me Ph | H | O |
| 39 | H | H | Et | 3-MeO Ph | H | O |
| 40 | H | H | Et | 4-F-Ph | H | O |
| 41 | H | H | Et | 4-Cl Ph | H | O |
| 42 | H | H | Et | 4-CF₃ Ph | H | O |
| 43 | H | H | Et | 4-Me Ph | H | O |
| 44 | H | H | Et | 4-Et Ph | H | O |
| 45 | H | H | Et | 4-MeO Ph | H | O |
| 46 | H | H | Et | 4-EtO Ph | H | O |
| 47 | H | H | Et | 2,3-di-F Ph | H | O |
| 48 | H | H | Et | 2,4-di-F Ph | H | O |
| 49 | H | H | Et | 3,4-di-F Ph | H | O |
| 50 | H | H | Et | 2,6-di-F Ph | H | O |
| 51 | H | H | Et | 2,3-di-Cl Ph | H | O |
| 52 | H | H | Et | 2,4-di-Cl Ph | H | O |
| 53 | H | H | Et | 3,4-di-Cl Ph | H | O |
| 54 | H | H | Et | 2,6-di-Cl Ph | H | O |
| 55 | H | H | Et | 2,3-di-Me Ph | H | O |
| 56 | H | H | Et | 2,4-di-Me Ph | H | O |
| 57 | H | H | Et | 3,4-di-Me Ph | H | O |
| 58 | H | H | Et | 2,6-di-Me Ph | H | O |
| 59 | H | H | Et | 2,3-di-MeO Ph | H | O |
| 60 | H | H | Et | 2,4-di-MeO Ph | H | O |
| 61 | H | H | Et | 3,4-di-MeO Ph | H | O |
| 62 | H | H | iPr | Ph | H | O |
| 63 | H | H | iPr | 2-F Ph | H | O |
| 64 | H | H | iPr | 2-Cl Ph | H | O |
| 65 | H | H | iPr | 2-Me Ph | H | O |
| 66 | H | H | iPr | 2-MeO Ph | H | O |
| 67 | H | H | iPr | 3-F-Ph | H | O |
| 68 | H | H | iPr | 3-Cl Ph | H | O |
| 69 | H | H | iPr | 3-CF₃ Ph | H | O |
| 70 | H | H | iPr | 3-Me Ph | H | O |
| 71 | H | H | iPr | 3-MeO Ph | H | O |
| 72 | H | H | iPr | 4-F-Ph | H | O |
| 73 | H | H | iPr | 4-Cl Ph | H | O |
| 74 | H | H | iPr | 4-CF₃ Ph | H | O |
| 75 | H | H | iPr | 4-Me Ph | H | O |
| 76 | H | H | iPr | 4-Et Ph | H | O |
| 77 | H | H | iPr | 4-MeO Ph | H | O |
| 78 | H | H | iPr | 4-EtO Ph | H | O |
| 79 | H | H | iPr | 2,3-di-F Ph | H | O |
| 80 | H | H | iPr | 2,4-di-F Ph | H | O |
| 81 | H | H | iPr | 3,4-di-F Ph | H | O |
| 82 | H | H | iPr | 2,3-di-F Ph | H | O |
| 83 | H | H | iPr | 2,3-di-Cl Ph | H | O |
| 84 | H | H | iPr | 2,4-di-Cl Ph | H | O |
| 85 | H | H | iPr | 2,6-di-Cl Ph | H | O |
| 86 | H | H | iPr | 3,4-di-Cl Ph | H | O |
| 87 | H | H | iPr | 2,3-di-Me Ph | H | O |
| 88 | H | H | iPr | 2,4-di-Me Ph | H | O |
| 89 | H | H | iPr | 2,3-di-Me Ph | H | O |
| 90 | H | H | iPr | 2,3-di-Me Ph | H | O |
| 91 | H | H | iPr | 2,3-di-MeO Ph | H | O |
| 92 | H | H | iPr | 2,4-di-MeO Ph | H | O |
| 93 | H | H | iPr | 3,4-di-MeO Ph | H | O |
| 94 | Me | H | Et | 4-MeO Ph | H | O |
| 95 | Me | H | Et | 4-MeO Ph | H | S |
| 96 | H | H | Et | 4-MeO Ph | H | S |
| 97 | H | H | Me | 4-Et Ph | H | S |
| 98 | H | F | Et | 4-MeO Ph | H | O |
| 99 | H | H | Et | 4-MeO Ph | F | O |
| 100 | H | H | Et | 4-F-Ph | H | S |
| 101 | H | H | Et | 4-Cl Ph | H | S |
| 102 | H | H | Et | 4-CF₃ Ph | H | S |
| 103 | H | H | Et | 4-Me Ph | H | S |
| 104 | H | H | Et | 4-MeO Ph | H | S |
| 105 | H | H | Et | 4-EtO Ph | H | S |

The particular process to be utilized in the preparation of the compounds of this invention depends upon the specific compound desired. Such factors as the selection of the specific X moiety, and the specific substituents possible at various locations on the molecule, all play a role in the path to be followed in the preparation of the specific compounds of this invention. Those factors are readily recognized by one of ordinary skill in the art.

In general, the compounds of this invention may be prepared by standard techniques known in the art and by known processes analogous thereto. For example, the compounds may be prepared according to methods described in U.S. Pat. No. 6,828,335, and U.S. application Ser. No. 13/375,878 filed on Dec. 2, 2011 which are incorporated by reference in its entirety. The present invention also encompasses indane acetic acid compounds and derivatives described in U.S. Pat. No. 7,112,597 in U.S. Pat. No. 8,541,618, and in U.S. Pat. No. 8,552,203, which are incorporated by references in their entirety. The present invention also encompasses indane acetic acid derivatives and their use described in US application publication number 2014/0086910, publication date Mar. 27, 2014 and in U.S. patent application Ser. No. 14/477,114, filed on Sep. 4, 2014, which are incorporated by references in their entirety.

A salt of a compound described in the present invention may be prepared in situ during the final isolation and purification of a compound or by separately reacting the purified compound in its free base form with a suitable organic or inorganic acid and isolating the salt thus formed. Likewise, when the compound described in the present invention contains a carboxylic acid moiety, (e.g., R═H), a salt of said compound may be prepared by separately reacting it with a suitable inorganic or organic base and isolating the salt thus formed. The term “pharmaceutically acceptable salt” refers to a relatively non-toxic, inorganic or organic acid addition salt of a compound of the present invention (see, e.g., Berge et al., J. Pharm. Sci. 66:1-19, 1977).

Representative salts of the compounds described in the present invention include the conventional non-toxic salts and the quaternary ammonium salts, which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, undecanoate, and the like.

Base salts include, for example, alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups in the conjugate base may be quaternized with alkyl halides, e.g., C₁₋₉ alkyl halides such as methyl, ethyl, propyl, and butyl chlorides, bromides and iodides; dialkyl sulfates like dimethyl, diethyl, and dibutyl sulfate; and diamyl sulfates, C₁₀₋₄₀ alkyl halides such as decyl, lauryl, myristyl and stearyl chlorides, bromides and iodides; or aralkyl halides like benzyl and phenethyl bromides. In some embodiments, the salts are alkali salt such as sodium or potassium salt or an adduct with an acceptable nitrogen base such as meglumine (N-Methyl-d-glucamine) salt.

The esters of the compounds described in the present invention are non-toxic, pharmaceutically acceptable esters, for example, alkyl esters such as methyl, ethyl, propyl, isopropyl, butyl, isobutyl, or pentyl esters. Additional esters such as, for example, methyl ester or phenyl-C₁-C₅ alkyl may be used. The compound described in the present invention may be esterified by a variety of conventional procedures including reacting the appropriate anhydride, carboxylic acid, or acid chloride with the alcohol group of the compounds described in the present invention compound. The appropriate anhydride may be reacted with the alcohol in the presence of a base to facilitate acylation such as 1,8-bis[dimethylamino]naphthalene or N,N-dimethylaminopyridine. An appropriate carboxylic acid may be reacted with the alcohol in the presence of a dehydrating agent such as dicyclohexylcarbodiimide, 1-[3-dimethylaminopropyl]-3-ethylcarbodiimide, or other water soluble dehydrating agents which are used to drive the reaction by the removal of water, and optionally, an acylation catalyst. Esterification may also be effected using the appropriate carboxylic acid in the presence of trifluoroacetic anhydride and optionally, pyridine, or in the presence of N, N-carbonyldiimidazole with pyridine. Reaction of an acid chloride with the alcohol may be carried out with an acylation catalyst such as 4-DMAP or pyridine. One skilled in the art would readily know how to successfully carry out these, as well as other methods of esterification of alcohols.

Additionally, sensitive or reactive groups on the compound described in the present invention may need to be protected and deprotected during any of the above methods for forming esters. Protecting groups in general may be added and removed by conventional methods well known in the art (see, e.g., Chapter One of T. W. Greene and P. G. M. Wuts, Protective Groups in Organic Synthesis; Wiley: New York, (1999)).

The compounds described in the present invention may contain one or more asymmetric centers, depending upon the location and nature of the various substituents desired. Asymmetric carbon atoms may be present in the (R) or (S) configuration. Preferred isomers are those with the absolute configuration, which produces the compound of described in the present invention with the more desirable biological activity. In certain instances, asymmetry may also be present due to restricted rotation about a given bond, for example, the central bond adjoining two aromatic rings of the specified compounds.

Substituents on a ring may also be present in either cis or trans form, and a substituent on a double bond may be present in either Z or E form.

It is intended that all isomers (including enantiomers and diastereomers), either by nature of asymmetric centers or by restricted rotation as described above, as separated, pure or partially purified isomers or racemic mixtures thereof, be included within the scope of the instant invention. The purification of said isomers and the separation of said isomeric mixtures may be accomplished by standard techniques known in the art.

As described herein, compounds of the invention may optionally be substituted with one or more substituents, such as are illustrated generally above, or as exemplified by particular classes, subclasses, and species of the invention. In general, the term “substituted” refers to the replacement of hydrogen radicals in a given structure with the radical of a specified substituent. Unless otherwise indicated, a substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

### C. EVALUATION OF BIOLOGICAL ACTIVITY OF COMPOUNDS

PPAR receptor agonist activity may be determined by conventional screening methods known to the skilled in the art. For example, methods described in U.S. Patent Application Publication No. 2007/0054907, 2008/0262047 and U.S. Pat. No. 7,314,879, which are incorporated by reference in their entireties.

The blood-brain barrier also eliminates lipophilic molecules by way of an active transport mechanism mediated by P-glycoprotein (P-gp). For a neurodegenerative disease therapeutic to be effective it has to achieve balance between passive diffusion in through the BBB, and active elimination out by the P-gp transporter, or other transporters. P-gp is an ATP-dependent, drug efflux pump for xenobiotic compounds with broad tissue distribution including the endothelia cells of the BBB. [Schinkel A H (April 1999). “P-Glycoprotein, a gatekeeper in the blood-brain barrier”. Advanced Drug Delivery Reviews 5 (36(2-3)): 179-194.]. P-gp activity can be measured in pre-clinical in vitro studies.

### D. ANIMAL STUDIES

The compounds described in the present invention may be tested in any animal model known to those skilled in the art. Exemplary animal models of neurotoxicity include, but are not limited to, transgenic mouse models, the EAE (experimental autoimmune encephalomyelitis, an animal model for MS; The MPTP or 6-OHDA toxin induced Parkinsons Disease Models, The murine blast neurotrauma model, which investigates the mechanistic linkage between blast exposure, CTE neuropathology, and neurobehavioral sequalae. It uses a compressed gas blast tube designed to accommodate wild-type C57BL/6 male mice and allowed free movement of the head and cervical spine to model typical conditions associated with military blast exposure.

Blood brain penetration studies can be carried out as described in recent publications and as is known by those skilled in the art. (See: Chang, K. L., et al. Influence of drug transporters and stereoselectivity on the brain penetration of pioglitazone as a potential medicine against Alzheimer's disease. Science Reports 2014, 5, 9000). In brief, A good measure of whether a small molecule penetrates the BBB and is not rapidly transported out, is the brain to plasma ratio of the drug. This is measured in pre-clinical animal models by determining plasma concentration vs time curves as in a standard pharmacokinetic study, and in addition harvesting brains and determining whole brain concentrations over time. The brain to plasma concentration ratio can them be determined at any time point, such as the Cₘₐₓ, or for the entire time curve (AUC, area under the curve). Clinically, FDG-PET can be used as a measure of the pharmacological effect of T3D-959, and an indirect measure of brain levels of drug.

For each model, the test result is compared with a control group that is not treated with the compounds described in the present invention. The treated animals are expected to demonstrate significant improvement in the performance of a variety of tests that measure steatosis, inflammation, fibrosis, dyslipidemia, and insulin resistance.

### E. PHARMACEUTICAL COMPOSITIONS

According to another aspect of the present invention, pharmaceutical compositions of compounds described herein are provided. In some embodiments, the pharmaceutical compositions further include a pharmaceutically acceptable carrier.

In some embodiments, the pharmaceutical compositions described herein may further include one or more additional therapeutic agents.

In one embodiment, the additional therapeutic agents are used to treat or prevent the following diseases:

### Alzheimer's Disease (AD)

### Huntington's Disease (HD)

### Parkinson's Disease (PD)

Amyotrophic lateral sclerosis (ALS)

### Frontal Temporal Dementia (FTD)

### Corticobasal Degeneration (CBD)

### Progressive Supranuclear Palsy (PSP)

Dementia with Lewy Bodies (LBD)

### Multiple Sclerosis (MS).

Exemplary additional therapeutic agents include, but are not limited to combination with: PPAR gamma agonists such as rosiglitazone and pioglitazone, metformin, pentoxifylline, vitamin E, selenium, omega-3 fatty acids and betaine; therapeutic agents used to treat Alzheimer's disease such as Donepezil, Rivastigmine, or Memantine; therapeutic agents to treat Parkinson's Disease (PD) such as L DOPA (levodopa), or dopamine agonists; therapeutic agents used to treat Amyotrophic Lateral Sclerosis (ALS) such as Edaravone (Radicut), or Riluzole; and therapeutic agents used to treat Multiple Sclerosis (MS) such as interferon beta, glatiramer acetate, mitoxantrone, natalizumab, fingolimod, teriflunomide, alemtuzumab, daclizumab, or ocrelizumab.

Based on well-known assays used to determine the efficacy for treatment of conditions identified above in mammals, and by comparison of these results with the results of known medicaments that are used to treat these conditions, the effective dosage of the compounds of this invention can readily be determined for treatment of each desired indication. The amount of the active ingredient (e.g., compounds) to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.0001 mg/kg to about 10 mg/kg, and preferably from about 0.001 mg/kg to about 10 mg/kg body weight per day. A unit dosage may contain from about 0.05 mg to about 500 mg of active ingredient, and may be administered one or more times per day. The daily dosage for administration by injection, including intravenous, intramuscular, subcutaneous, intranasal and parenteral injections, and use of infusion techniques may be from about 0.0001 mg/kg to about 10 mg/kg. The daily rectal dosage regimen may be from 0.0001 mg/kg to 10 mg/kg of total body weight. The transdermal concentration may be that required to maintain a daily dose of from 0.0001 mg/kg to 10 mg/kg. The daily intranasal dosage regimen may be from 0.0001 mg/kg to 10 mg/kg of total body weight.

Of course, the specific initial and continuing dosage regimen for each patient will vary according to the nature and severity of the condition as determined by the attending diagnostician, the activity of the specific compound employed, the age of the patient, the diet of the patient, time of administration, route of administration, rate of excretion of the drug, drug combinations, and the like. The desired mode of treatment and number of doses of a compound of the present invention may be ascertained by those skilled in the art using conventional treatment tests.

The compounds of this invention may be utilized to achieve the desired pharmacological effect by administration to a patient in need thereof in an appropriately formulated pharmaceutical composition. A patient, for the purpose of this invention, is a mammal, including a human, in need of treatment for a particular condition or disease. Therefore, the present invention includes pharmaceutical compositions which include a pharmaceutically acceptable carrier and a therapeutically effective amount of a compound. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A therapeutically effective amount of a compound is that amount which produces a result or exerts an influence on the particular condition being treated. The compounds described herein may be administered with a pharmaceutically-acceptable carrier using any effective conventional dosage unit forms, including, for example, immediate and timed release preparations, orally, parenterally, topically, intranasally or the like.

For oral administration, the compounds may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soybean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

The compounds of this invention may also be administered parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quaternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The compounds of this invention may also be administered intranasally, as dosage of the compound in a physiologically acceptable diluent with a pharmaceutical carrier which may be a sterile liquid or mixture of liquids such as water, saline, aqueous dextrose and related sugar solutions; an alcohol such as ethanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug (e.g., compound) with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, or trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC—CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (includes but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, octoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrilin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

The compounds described herein may be administered as the sole pharmaceutical agent or in combination with one or more other pharmaceutical agents where the combination causes no unacceptable adverse effects. For example, compounds of this invention can be combined with known anti-oxidants, anti-obesity agents, insulin sensitizers, anti-fibrotics, anti-dyslipidemics, and the like, as well as with admixtures and combinations thereof.

The compounds described herein may also be utilized, in free acid or base form or in compositions, in research and diagnostics, or as analytical reference standards, and the like. Therefore, the present invention includes compositions which include an inert carrier and an effective amount of a compound identified by the methods described herein, or a salt or ester thereof. An inert carrier is any material which does not interact with the compound to be carried and which lends support, means of conveyance, bulk, traceable material, and the like to the compound to be carried. An effective amount of compound is that amount which produces a result or exerts an influence on the particular procedure being performed.

The compounds may be administered to subjects by any suitable route, including orally (inclusive of administration via the oral cavity), parenterally, by inhalation spray, topically, transdermally, rectally, nasally, sublingually, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, parenterally, transdermally or by inhalation spray.

It should also be understood that a specific dosage and treatment regimen for any particular patient will depend upon a variety of factors, including the activity of the specific compound employed, the age, body weight, general health, gender, diet, time of administration, rate of excretion, drug combination, and the judgment of the treating physician and the severity of the particular disease being treated. The amount of a compound of the present invention in the composition will also depend upon the particular compound in the composition.

The following examples are presented to illustrate the invention described herein, but should not be construed as limiting the scope of the invention in any way.

Capsule Formulation

A capsule formula is prepared from:

| | |
|---|---|
| Compound of this invention | 10 mg |
| Starch | 109 mg |
| Magnesium stearate | 1 mg |

The components are blended, passed through an appropriate mesh sieve, and filled into hard gelatin capsules.

Tablet Formulation

A tablet is prepared from:

| | |
|---|---|
| Compound of this invention | 25 mg |
| Cellulose, microcrystalline | 200 mg |
| Colloidal silicon dioxide | 10 mg |
| Stearic acid | 5.0 mg |

The ingredients are mixed and compressed to form tablets. Appropriate aqueous and non-aqueous coatings may be applied to increase palatability, improve elegance and stability or delay absorption.

Sterile IV Solution

A mg/mL solution of the desired compound of this invention is made using sterile, injectable water, and the pH is adjusted if necessary. The solution is diluted for administration with sterile 5% dextrose and is administered as an IV infusion.

Intramuscular Suspension

The following intramuscular suspension is prepared:

| | |
|---|---|
| Compound of this invention | 50 mg/mL |
| Sodium carboxymethylcellulose | 5 mg/mL |
| TWEEN 80 | 4 mg/mL |
| Sodium chloride | 9 mg/mL |
| Benzyl alcohol | 9 mg/mL |

The suspension is administered intramuscularly.

Hard Shell Capsules

A large number of unit capsules are prepared by filling standard two-piece hard galantine capsules each with powdered active ingredient, 150 mg of lactose, 50 mg of cellulose, and 6 mg of magnesium stearate.

Soft Gelatin Capsules

A mixture of active ingredients in a digestible oil, such as soybean oil, cottonseed oil, or olive oil, is prepared and injected by means of a positive displacement pump into molten gelatin to form soft gelatin capsules containing the active ingredient. The capsules are washed and dried. The active ingredient can be dissolved in a mixture of polyethylene glycol, glycerin and sorbitol to prepare a water miscible medicine mix.

Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin, and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Intranasal Formulation

These are liquid intranasal dosage forms made by conventional and novel processes. The active ingredient is mixed in a liquid containing ingredient such as sugar, gelatin, pectin.

### F. METHODS OF USE

Treatment of neurodegenerative diseases including:

### Alzheimer's Disease (AD)

### Huntington's Disease (HD)

### Parkinson's Disease (PD)

Amyotrophic lateral sclerosis (ALS)

### Frontal Temporal Dementia (FTD)

### Corticobasal Degeneration (CBD)

### Progressive Supranuclear Palsy

Dementia with Lewy Bodies (LBD or DLB)

### Multiple Sclerosis (MS).

According to one aspect of the present invention, methods of preventing or treating neurodegenerative diseases and the cited conditions are provided. The methods include administering to a subject in need of such treatment an effective amount of a compound of the present invention. In some embodiments, the compound is administered intravenously, orally, buccally, transdermally, rectally, nasally, optically, intrathecally, or intra-cranially.

In another embodiment, the compounds of the present invention may be administered in combination with one or more additional therapeutic agent. Exemplary additional therapeutic agents include, but are not limited to Dexmedetomidine (Precedex), Ephedrine, Epinephrine, Norepinephrine, Dopamine, Remifentanil, Sevoflurane, Melatonin (N-acetyl-5-methoxytryptamine), Atorvastatin, Donepezil, Sugammadex, Methylprednisolone, Human Growth Hormone, Topiramate, Phenytoin, Duloxetine, Sertraline, Mannitol, Memantine, Buspirone, Rivastigmine, Epoprostenol (Prostacyclin), Atomoxetine (Strattera), Amantadine, Propranolol, Citalopram (Celexa), Erythropoietine (recombinant human erythropoietine), Venlafaxine, Ondansetron, Oxycyte, Armodafinil, Modafinil, Enoxaparin, Cycloserine, Citralopram, Paracetamol, Carbamazepine, Tizanidine HCL, OnabotulinumtoxinA, Sildenafil, Methylphenidate, Valine, Amitriptyline, Prazosin hydrochloride, Cerebrolysin (nerve growth factor), Paroxetine, Avlocardyl, Escitalopram, Prazosine, Atorvastatin, Sertraline, Topiramate, Mirtazapine, Mifepristone, Rapamycin, Venlafaxine, Eszopiclone, Fluoxetine, Oxytocin, Prazosin, Excitalopram, Levetiracetam, MDMA (3,4-Methylenedioxymethamphetamine), N-acetylcysteine, Dronabinol, Mifepristone, Naltrexone, Ifenprodil tartrate, Resperidone, D-Cycloserine, Hydrocortisone, Tramadol, Doxazosin, Riluzole, Yohimbine, Ketamine, Fluoxetine, Escitalopram, Quetiapine, Clonazepam, Geodon (Ziprasidone), Clopidogrel, Atomoxetine, Levosimendan, [Reperfusion Injury] Viaspan, Mycophenolic Acid, Thymoglobulin, Atorvastatin, Rosiglitazone, Dipyridamole, Eplerenone, Aldosterone, Eculizumab, Tacrolimus, N-Acetylcysteine, Antithrombin-III, Infliximab, Apotranserrin, Melatonin, Nitric oxide, Ciclosporin, Bendavia, Treprostinil, Cylexin, Vitamin C, Simvastatin, Sevoflurane, Metformin, Celebrex, [Spinal Cord Injury] Zoledronic Acid, Pregabalin, Minocycline, L-Carnitine, Ibuprofen, Lithium Carbonate, Dalfampridine, Minocycline, Growth Hormone, Lexapro, Cethrin (BA-210), Amitriptyline, Cannabis, Fampridine, Denosumab, Riluzole, Vardenafil, Venalafaxine hydrochloride, [Parkinsons Disease] Rasagiline, Memantine, Domperidone, Levodopa, Ipratropium, Sinemet, carbidopa, Requip, Mirapex, Neupro, Symmetrel, Artane, Cogentin, Eldepryl, Azilect, Tasmar, Comtan [Huntingtons Disease] Olanzapine, Tiapridal, Xenazine, Dmebon, Minocyclin, PF-0254920, VX15/2503, Pridopidine, Divalproex, EGCG, Bupropion, Citalopram, Atomoxetine, ACR16, Memantine, SD-809, Cysteamine (RP103), tetrabenazine, [ALS] Thalidomide, CK-2017357, Tauroursodexycholic acid (TUDCA), NP001, Insulin-like Growth Factor-1, Glatiramer Acetate, Rasagiline, Tamoxifen, SB-509, Tocilizumab, Dexpramipexole, Cimetidine, Excitalopram (Lexapro), Arimoclomol, KNS-760704, Ceftriaxone, Ebixa, MCI-186, Pioglitazone, Tretinoin, Rasagiline, Dexpramipexole, Riluzole, ONO2506PO, Olesoxime, AVP-923, Olanzapine, Sodium Valproate, Mecobalamin, TRO19622, MYOBLOC, [MS] Copaxone, Glatiramer Acetate (GTR), L-Carnitine, Teriflunomide, Betaseron, Lipoic Acid, Sativex, THC, Sativex, Acthar, ACTH, Teriflunomide, Cladribine, Ocrelizumab, Rebif, Fingolimod, Armodafinil, RPC1063, Aspirin, Pregabalin, Paroxetine, Dronabinol, Methylprednisolone, Alemtuzumab, Tysabri, Memantine, Dimethyl Fumarate, Vitamin D3, Cholecalciferol, Sunphenon, Naltrexone, FTY720, Simvastatin, Laquinimod, Avonex, AVP-923, Fampridine, EGCG, [HIV Dementia] Zidovudine, Stavudine, Abacavir, Didanosine, OPC-14117, Methylphenidate, CPI-1189, Nimodipine, Atorvastatin, Cenicriviroc, Maraviroc, Darunavir, Thioctic Acid, Fluconazole, Paroxetine, Rivastigmine, Deprenyl, Saquinavir, Nevirapine, Lamivudine, Emtricitabine, Tenofovir, Delavirdine mesylate, Efavirenz, Ritonavir, Enfuvirtide, Nelfinavir, Amprenavir, Tipranavir, Lopinavir, Atazanavir, Tenofovir, Disoproxil Fumarate, Darunavir, Fosamprenavir, Etravirine, Trizivir, Maraviroc, Raltegravir, Metformin, and PPAR gamma agonists such as rosiglitazone and pioglitazone. The compounds described herein may be administered in combination with one or more further medicaments of use for the treatment or prevention of the listed conditions and disease.

Depending on the individual medicaments utilized in a combination therapy for simultaneous administration, they may be formulated in combination (where a stable formulation may be prepared and where desired dosage regimes are compatible) or the medicaments may be formulated separately (for concomitant or separate administration through the same or alternative routes).

In some embodiments, the subject of the present invention possesses one or more risk factors for developing disease selected from a family history of the disease; Obesity, Insulin resistance and Type 2 Diabetes, and Metabolic syndrome.

In some embodiments, the subject of the present invention may be examined by FDG-PET (fluoro deoxyglucose positron emission tomography) to demonstrate brain penetration of the indane acetic acid PPAR agonist, and supporting penetration of the compound through the BBB (blood brain barrier).

### G. EXAMPLES

Embodiments of the present invention will now be described by way of example only with respect to the following non-limiting examples.

In general, the compounds of this invention may be prepared by standard techniques known in the art and by known processes analogous thereto. For example, the compounds may be prepared according to methods described in U.S. Pat. No. 6,828,335, and U.S. application Ser. No. 13/375,878 filed on Dec. 2, 2011 which are incorporated by reference in its entirety.

### Example 1

### Ethyl [(1S)-5-hydroxy-2,3-dihydro-1H-inden-1-yl]acetate

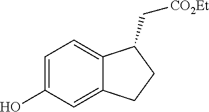 [SMILES: CCOC(=O)C[C@@H]1CCc2cc(O)ccc21]

Prepared in six steps from 5-methoxy indanone as described in US68283335.

### Example 2

### 2-[5-ethyl-2-(4-methoxyphenyl)-1,3-oxazol-4-yl]ethanol

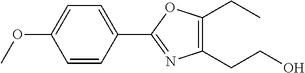 [SMILES: CCc1oc(-c2ccc(OC)cc2)nc1CCO]

Prepared from L-aspartic acid β-methyl ester hydrochloride, 4-methoxy benzoyl chloride and proprionic anhydride as generally described in US68283335.

### Example 3

### 2-[2-(4-methoxyphenyl)-5-methyl-1,3-oxazol-4-yl]ethanol

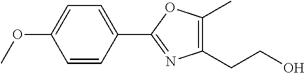 [SMILES: COc1ccc(-c2nc(CCO)c(C)o2)cc1]

Prepared from L-aspartic acid β-methyl ester hydrochloride, 4-methoxy benzoyl chloride and acetic anhydride as generally described in US68283335.

### Example 4

### 2-[5-Ethyl-2-(4-methylphenyl)-1,3-oxazol-4-yl]ethanol

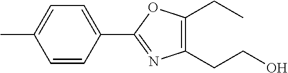 [SMILES: CCc1oc(-c2ccc(C)cc2)nc1CCO]

Prepared from L-aspartic acid β-methyl ester hydrochloride, p-toluoyl chloride and proprionic anhydride as generally described in US68283335.

### Example 5

### 2-[5-Methyl-2-(4-methylphenyl)-1,3-oxazol-4-yl]ethanol

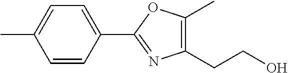 [SMILES: Cc1ccc(-c2nc(CCO)c(C)o2)cc1]

Prepared as from L-aspartic acid β-methyl ester hydrochloride, p-toluoyl chloride and acetic anhydride as described in US68283335.

### Example 6

### 2-[5-Ethyl-2-(4-ethylphenyl)-1,3-oxazol-4-yl]ethanol

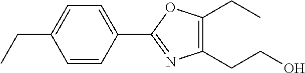 [SMILES: CCc1ccc(-c2nc(CCO)c(CC)o2)cc1]

Prepared from L-aspartic acid β-methyl ester hydrochloride, 4-ethyl benzoyl chloride and proprionic anhydride as generally described in US68283335.

### Example 7

### 2-[2-(4-Ethylphenyl)-5-methyl-1,3-oxazol-4-yl]ethanol

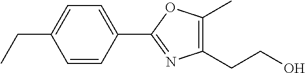 [SMILES: CCc1ccc(-c2nc(CCO)c(C)o2)cc1]

Prepared from L-aspartic acid β-methyl ester hydrochloride, 4-ethyl benzoyl chloride and acetic anhydride as generally described in US68283335.

### Example 8

### 2-(5-ethyl-2-(4-methoxyphenyl)oxazol-4-yl)ethyl benzenesulfonate

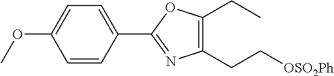

The intermediate from Example 2 (400.8 g), 15.0 g trimethylamine hydrochloride and 3.2 L dichloromethane was added to a 22 L reactor. The reaction mixture was stirred and cooled to 3.8° C. 680 mL of triethylamine was then added to the reactor. Benzenesulfonyl chloride (400 g) is slowly added to the reactor while maintaining the temperature below 12° C. The reaction was cooled to between 5° C. and 10° C. for three hours and then heated to 20° C. The contents of the reactor were stirred overnight at 24° C. Additional 3.2 L of dichloromethane was added to the reactor. The mixture was cooled to 5.0° C. and 205 mL 3-dimethylamino-1-propylamine was added. The mixture is stirred at 4.8° C. for 16 minutes. An aqueous citric acid solution (3 L of 1M) was slowly added to the reactor so as to maintain the temperature below 16° C. The resulting mixture was heated to 20° C. and stirred for 10 minutes. The phases were separated, and the organics were washed with 3 L of 1M citric acid solution, 3 L saturated sodium bicarbonate solution, 3 L brine solution, dried with magnesium sulfate, filtered and concentrated. The residue was treated with n-heptane and concentrated to give 542 g of crude 2-(5-ethyl-2-(4-methoxyphenyl)oxazol-4-yl)ethyl benzenesulfonate.

### Example 9

### (S)-Ethyl 2-(5-(2-(5-ethyl-2-(4-methoxyphenyl)oxazol-4-yl)ethoxy)-2,3-dihydro-1H-inden-1-yl)acetate

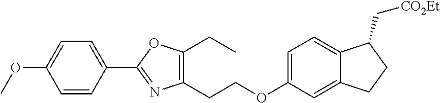 [SMILES: CCOC(=O)C[C@@H]1CCc2cc(OCCc3nc(-c4ccc(OC)cc4)oc3CC)ccc21]

A 22 L reactor was charged with 302.3 g of ethyl [(1 S)-5-hydroxy-2,3-dihydro-1H-inden-1-yl]acetate (Example 1), 539.3 g crude 2-(5-ethyl-2-(4-methoxyphenyl)oxazol-4-yl)ethyl benzenesulfonate (Example 8) and 3.4 L acetonitrile. The mixture was stirred until all of the solids dissolved; then, 670.6 g cesium carbonate was added. The mixture is heated to 70° C. and held 16 hours. An additional charge of 60.2 g of compound from Example 1 was added to the reactor. The mixture was heated to 70° C. for one hour and additional cesium carbonate (316.9 g) was added and heating was continued for 2.5 hours at 70° C. The reaction mixture was cooled to 24° C. and 4 L n-heptane, 2.4 L USP water, 2.4 L brine solution and 4 L ethyl acetate was charged to the reactor. The biphasic mixture was stirred for 5 minutes, then allowed to separate. The organic layer was washed with 2×2.4 L 5% sodium hydroxide solution and 2.4 L USP water, and 2.4 L brine. The solvent is removed via rotary evaporation until solids precipitate. Addition of 7.7 L n-heptane and stirring produced a slurry, which was filtered, and the filter cake was rinsed with the filtrate and then with 2.4 L n-heptane. The product air dried and then dried in a vacuum oven at 40° C. to give (S)-ethyl 2-(5-(2-(5-ethyl-2-(4-methoxyphenyl)oxazol-4-yl)ethoxy)-2,3-dihydro-1H-inden-1-yl)acetate as an off white solid.

### Example 10

### (S)-2-(5-(2-(5-ethyl-2-(4-methoxyphenyl)oxazol-4-yl)ethoxy)-2,3-dihydro-1H-inden-1-yl)acetic acid

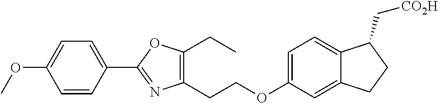 [SMILES: CCc1oc(-c2ccc(OC)cc2)nc1CCOc1ccc2c(c1)CC[C@H]2CC(=O)O]

A 22 L flask was charged with 478.9 g of (S)-ethyl 2-(5-(2-(5-ethyl-2-(4-methoxyphenyl)oxazol-4-yl)ethoxy)-2,3-dihydro-1H-inden-1-yl)acetate (Example 9) and 1.2 L ethanol and cooled to 20° C. To the 22 L flask was charged 1.6 L of 1N sodium hydroxide solution. The reaction mixture was heated to 65° C. for 30, then cooled to 25° C., and concentrated to an oil. A new reaction flask was charged with 4.8 L USP water and 1.9 L 1N hydrochloric acid solution, vigorously stirred and cooled to 23° C. The product oil was added to the solution via an addition funnel. The resulting suspension is stirred at approximately 23° C., and the pH is checked: 1.6 (target ≤2). The solids were filtered and then washed with the mother liquor. The solids were washed with 3 L USP water and then with 1.9 L 1:1 ethanol SDA-2B:water. The filter cake was air dried for 4 hours and is then transferred to a vacuum oven. The solid was dried under vacuum at 45° C. until a constant mass was achieved, producing (S)-2-(5-(2-(5-ethyl-2-(4-methoxyphenyl)oxazol-4-yl)ethoxy)-2,3-dihydro-1H-inden-1-yl)acetic acid as an off white solid.

### Example 11

### Sodium (S)-2-(5-(2-(5-ethyl-2-(4-methoxyphenyl)oxazol-4-yl)ethoxy)-2,3-dihydro-1H-inden-1-yl)acetate

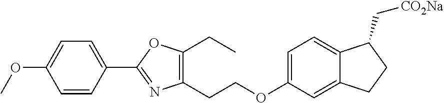

A 22 L reactor was charged with 3.8 L ethanol. Agitation was started, and the reactor was charged successively with 288.2 g sodium ethoxide solution (20.1% in ethanol) and with 378.4 g of (S)-2-(5-(2-(5-ethyl-2-(4-methoxyphenyl)oxazol-4-yl)ethoxy)-2,3-dihydro-1H-inden-1-yl)acetic acid (Example 10). The reaction mixture was heated to 40° C. for ˜20 minutes (until all solids are dissolved), and pH was checked (target pH 9-10).

The solution was filtered through a 10 micron filter membrane, returned to the reactor and heated to 40° C. The reactor was then charged with 3.4 L of filtered methyl t-butyl ether at such a rate that the temperature of the product solution is maintained at 40° C. throughout. The mixture is then seeded with 0.5 g Example 10 compound, and held at 42° C. for 40 minutes. An additional 3.4 L of filtered methyl t-butyl ether was added. The suspension was heated to 55° C. for 65 minutes. The suspension was cooled to 20-25° C. overnight then to 14° C. the next morning. The product was filtered under a nitrogen blanket, washed with 1.3 L filtered methyl t-butyl ether and dried to constant mass in a vacuum oven at 40° C. The bulk product was milled using a Comil with a 10 mesh sieve. The product is dried in a humidified environment at 40° C. NMR analysis showed ≤0.5% of ethanol by weight. Final product sodium (S)-2-(5-(2-(5-ethyl-2-(4-methoxyphenyl)oxazol-4-yl)ethoxy)-2,3-dihydro-1H-inden-1-yl)acetate was further dried at 45° C. under vacuum to obtain 306 g as a fine white solid.

### Example 12

### (S)-2-(5-(2-(2-(4-methoxyphenyl)-5-methyloxazol-4-yl)ethoxy)-2,3-dihydro-1H-inden-1-yl)acetic acid

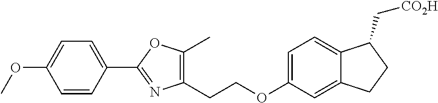 [SMILES: COc1ccc(-c2nc(CCOc3ccc4c(c3)CC[C@H]4CC(=O)O)c(C)o2)cc1]

(S)-Ethyl 2-(5-hydroxy-2,3-dihydro-1H-inden-1-yl)acetate from Example 1 and 2-(2-(4-methoxyphenyl)-5-methyloxazol-4-yl)ethanol from Example 3 were combined and reacted as in Examples 8, 9 and 10 to give (S)-2-(5-(2-(2-(4-methoxyphenyl)-5-methyloxazol-4-yl)ethoxy)-2,3-dihydro-1H-inden-1-yl)acetic acid as an off white solid.

### Example 13

### (S)-2-(5-(2-(5-ethyl-2-p-tolyloxazol-4-yl)ethoxy)-2,3-dihydro-1H-inden-1-yl)acetic acid

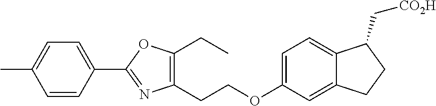 [SMILES: CCc1oc(-c2ccc(C)cc2)nc1CCOc1ccc2c(c1)CC[C@H]2CC(=O)O]

(S)-Ethyl 2-(5-hydroxy-2,3-dihydro-1H-inden-1-yl)acetate from Example 1 and 2-(5-ethyl-2-p-tolyloxazol-4-yl)ethanol from Example 4 were combined and reacted as in Examples 8, 9 and 10 to give (S)-2-(5-(2-(5-ethyl-2-p-tolyloxazol-4-yl)ethoxy)-2,3-dihydro-1H-inden-1-yl)acetic acid as an off white solid.

### Example 14

### (S)-2-(5-(2-(5-methyl-2-p-tolyloxazol-4-yl)ethoxy)-2,3-dihydro-1H-inden-1-yl)acetic acid

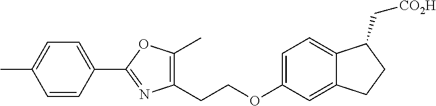 [SMILES: Cc1ccc(-c2nc(CCOc3ccc4c(c3)CC[C@H]4CC(=O)O)c(C)o2)cc1]

(S)-Ethyl 2-(5-hydroxy-2,3-dihydro-1H-inden-1-yl)acetate from Example 1 and 2-(5-methyl-2-p-tolyloxazol-4-yl)ethanol from Example 5 were combined and reacted as described in Examples 8, 9 and 10 to give (S)-2-(5-(2-(5-methyl-2-p-tolyloxazol-4-yl)ethoxy)-2,3-dihydro-1H-inden-1-yl)acetic acid as an off white solid.

### Example 15

### (S)-2-(5-(2-(5-ethyl-2-(4-ethylphenyl)oxazol-4-yl)ethoxy)-2,3-dihydro-1H-inden-1-yl)acetic acid

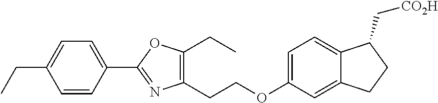 [SMILES: CCc1ccc(-c2nc(CCOc3ccc4c(c3)CC[C@H]4CC(=O)O)c(CC)o2)cc1]

(S)-Ethyl 2-(5-hydroxy-2,3-dihydro-1H-inden-1-yl)acetate from Example 1 and 2-(5-ethyl-2-(4-ethylphenyl)oxazol-4-yl)ethanol from Example 6 were combined and reacted as described in Examples 8, 9 and 10 to give (S)-2-(5-(2-(5-ethyl-2-(4-ethylphenyl)oxazol-4-yl)ethoxy)-2,3-dihydro-1H-inden-1-yl)acetic acid as an off white solid.

### Example 16

### (S)-2-(5-(2-(5-ethyl-2-(4-ethylphenyl)oxazol-4-yl)ethoxy)-2,3-dihydro-1H-inden-1-yl)acetic acid

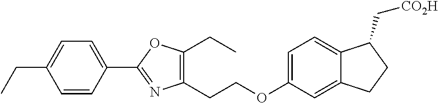 [SMILES: CCc1ccc(-c2nc(CCOc3ccc4c(c3)CC[C@H]4CC(=O)O)c(CC)o2)cc1]

(S)-Ethyl 2-(5-hydroxy-2,3-dihydro-1H-inden-1-yl)acetate from Example 1 and 2-(2-(4-ethylphenyl)-5-methyloxazol-4-yl)ethanol from Example 7 were combined and reacted as described in Examples 8, 9 and 10 to give (S)-2-(5-(2-(2-(4-ethylphenyl)-5-methyloxazol-4-yl)ethoxy)-2,3-dihydro-1H-inden-1-yl)acetic acid as an off white solid.

### Example 17

### Human PPAR Activation by Compound of Example 11 in Transient Transfection Study

The table summarize the results of studies performed in transfected CV-1 cells with three different lots of Compound of Example 11. These results showed an average EC₅₀ for activation of the human gamma subtype of 297 nM with a 74% maximal response. In similar experiments rosiglitazone had a human gamma subtype EC₅₀ of 130 nM. The average EC₅₀ for activation of the human δ subtype was 19 nM with a 76% maximal response. In similar experiments GW501516 had a human δ subtype EC₅₀ of 1.3 nM. The average EC₅₀ for activation of human alpha subtype was 530 nM with a significantly reduced maximal response. These results demonstrate that (S)-2-(5-(2-(5-ethyl-2-(4-methoxyphenyl)oxazol-4-yl)ethoxy)-2,3-dihydro-1H-inden-1-yl)acetate is a potent, selective agonist of the PPARδ and PPARγ subtypes, with a 15-fold greater potency in activating the human PPARδ subtype over the human PPARγ subtype and about a 30-fold selectivity over the human PPAR+balpha subtype.

Summary of Human PPAR Activation by
Compound of Example 11 in Transient Transfection Studies

| | Material | Use | PPARγ | PPARδ | PPARα |
|---|---|---|---|---|---|
| EC₅₀ (nM) | Lot A | Non GMP | 220 | 15 | 488 |
| | Lot B | GLP Nonclinical Studies | 270 | 27 | 750 |
| | Lot C | cGMP material | 400 | 16 | 360 |

| | | | |
|---|---|---|---|
| Average EC₅₀ (nM) | 297 | 19 | 530 |
| Average % maximal response | 74 | 76 | 49 |
| Peak effect | 29.49 | 7.68 | 1.87 |

### Example 18

### Effect of P-gp Inhibitor Verapamil on Caco-2 Permeability of Compound of Example 11

The human Caco-2 permeability of Compound of Example 11 was evaluated. High permeability of (S)-2-(5-(2-(5-ethyl-2-(4-methoxyphenyl)oxazol-4-yl)ethoxy)-2,3-dihydro-1H-inden-1-yl)acetate was observed in the absence of a P-glycoprotein (P-gp) inhibitor (Papparent=1144 nm/sec); no significant change in permeability was observed in the presence of the P-gp inhibitor (verapamil). These results indicate that (S)-2-(5-(2-(5-ethyl-2-(4-methoxyphenyl)oxazol-4-yl)ethoxy)-2,3-dihydro-1H-inden-1-yl)acetate is not a substrate for P-gp.

### Example 19

### Determination of Brain to Plasma Ratios for Compound of Example 11

Over 98% of drugs in clinical development for all diseases fail to adequately penetrate the blood brain barrier (BBB) to provide adequate brain exposure. For compounds of the present invention to be effective in treating neurotoxicity or neurodegeneration, it must have an ability to cross the BBB and penetrate the brain. To assess the ability of compounds of the present invention to cross the BBB, the pharmacokinetics and brain-to-plasma ratio of compound of Example 11, (Sodium (5)-2-(5-(2-(5-ethyl-2-(4-methoxyphenyl)oxazol-4-yl)ethoxy)-2,3-dihydro-1H-inden-1-yl)acetate was evaluated after oral dosing in male Sprague-Dawley rats. The test compound was dosed at 3 mg/kg from normal saline. Plasma and brain levels were determined by LC-MS/MS at pre-determined time points. Pharmacokinetic parameters were estimated by a non-compartmental model using WinNonlin v5.3 software. After oral dosing of test compound at 3 mg/kg, plasma Cₘₐₓ values of 1547±248 ng/mL were reached at 5 hours post dose. The average plasma half-life was 3.33 hours. The plasma exposure as measured by AUCₗₐₛₜ was 9569±1190 hr*ng/m L. Brain/Plasma ratios were found to be 0.361±0.142, 0.220±0.033, 0.171±0.011, 0.328±0.154, and 0.350±0.077 at 1, 3, 5, 7, and 12 hours, respectively. Results from the 15 rat experiment are shown in table form below.

Rat Brain Penetrance Pharmacokinetics
of Compound of Example 11

| | | | Brain | | | |
|---|---|---|---|---|---|---|
| Time | | Plasma | Tissue | | | |
| point | | Conc. | Conc. | B/P | Average | |
| (hr) | Rat# | (ng/mL) | (ng/g) | Ratio | (ng/g) | SD |
| 1.0 | 840 | 453 | 161 | 0.36 | 0.361 | 0.142 |
| | 841 | 199 | 101 | 0.51 | | |
| | 842 | 1020 | 226 | 0.22 | | |
| 3.0 | 843 | 910 | 234 | 0.26 | 0.220 | 0.033 |
| | 844 | 1270 | 269 | 0.21 | | |
| | 845 | 1430 | 274 | 0.19 | | |
| 5.0 | 846 | 1260 | 208 | 0.17 | 0.171 | 0.011 |
| | 847 | 1340 | 247 | 0.18 | | |
| | 848 | 2040 | 333 | 0.16 | | |
| 7.0 | 849 | 1240 | 203 | 0.16 | 0.328 | 0.154 |
| | 850 | 368 | 130 | 0.35 | | |
| | 851 | 462 | 216 | 47.00 | | |
| 12.0 | 852 | 345 | 104 | 0.30 | 0.350 | 0.077 |
| | 853 | 259 | 114 | 0.44 | | |
| | 854 | 377 | 117 | 0.31 | | |

### Example 20

### FDG-PET Data from Clinical Study with Compound of Example 11

A total of 36 subjects were enrolled into a two week long treatment study: 9 subjects randomized into 3 mg and 10 mg, 10 subjects randomized into 30 mg and 8 subjects randomized into 90 mg. The average age was 75 years with more than half of the subjects ranging between ages 65 to 84. Males and females were equally represented across all dose levels. The majority were non-Hispanic and of white origin. One half of the enrolled subjects carried one or two copies of the E4 allele for APOE genotype. FDG-PET scans were obtained at three clinical sites as described in the T3D959-201 Clinical Protocol. PET scans were obtained at baseline (BL) and again at end of treatment (EOT) for patients in all four dosage groups of Compound of Example 11 (3 mg, 10 mg, 30 mg, and 90 mg). The imaging protocol developed for the AD Neuroimaging Initiative (ADNI2) was used to collect the data. Overnight fasted subjects (blood glucose <180 mg/dL) received IV injection of 5 mCi [F18] fluoro-deoxyglucose as a single bolus. Subjects were instructed to lay supine with eyes open and forward. Thirty minutes after dosing, six 5-minute (total 30-min) emission scans were acquired. FDG-PET measurements obtained are relative to two reference regions, Whole Brain (WB) and cerebral White Matter (WM), for the computation of the changes in Relative Cerebral Metabolic Rate for glucose over the dosing period or: Δ R CMRgI (EOT-BL). The key primary outcome, was a global index, (sROI index) calculated from the average bq/voxel reading over an empirically pre-specified statistical Region of Interest (sROI), known to be affected by AD, which is normalized by the average bq/voxel for an empirically pre-specifed statistic ROI that is relatively spared. Change in the sROI index from BL to EOT is reported as Δ sROI. A second outcome was the determination of Δ R CMRgI (EOT-BL) for four pre-specified known AD-affected regions of interest (ROIs): 1) Posterior Cingulate (PC), 2) Precuneus (PreC), 3) Bilateral Middle Temporal Gyrus (BMTG), and 4) Right Inferior Parietal Lobule (RIPL). The final main outcome was a exploratory voxel-wise analysis of the whole brain to identify Regions of Statistically Significant Differences (ROSD) for Δ R CMRgI (EOT-BL) with uncorrected p<0.005. The voxel-wise analysis results are presented as slice by slice statistical map display superimposed on the anatomical T1 MR images with ROSDs highlighted in yellow. The R CMRgI values referred to in this report are calculated as the ratio of the average of the bq/voxel reading for each voxel, over each ROI, and divided by the average bq/voxel over the reference region used, e.g., WB or WM.

Results from the study suggest the following:

Compound of Example 11 (T3D-959) penetrates the Blood Brain Barrier (BBB) even at the lowest 3 mg. This conclusion comes from the voxel-wise (SPM) analysis and assumes that the findings are not solely attributable to small number of subjects, or the short test-retest interval.

T3D-959 altered the glucose metabolism in the brain. Increases and decreases in relative regional glucose metabolism (Δ R CMRgI (EOT-BL)) were observed over the treatment period. Table 4 below shows multiple regions of the brain with positive A R CMRgI (EOT-BL) Relative to Average Whole Brain for the 90 mg dose group. These are regions which are responding better to T3D-959 than the average whole brain.

Brain Regions with Positive Δ R
CMRgl (EOT-BL) Relative to Average
Whole Brain for the 90 mg Group (ANOVA design)

| | | Talairach | | |
|---|---|---|---|---|
| | | Coordinates | | |
| Brain Region | P value | (x,y,z) | | |
| Putamen_R* | 6.23E−07 | 32 | −13 | 8 |
| Vermis_3 | 3.14E−06 | −2 | −35 | −5 |
| Putamen_R | 3.15E−06 | 32 | −11 | 4 |
| ParaHippocampal_R | 5.05E−06 | 24 | −5 | −20 |
| Putamen_R | 7.42E−06 | 32 | −11 | 12 |
| Caudate_R | 3.08E−05 | 10 | 7 | −10 |
| Hippocampus_L | 3.58E−05 | −26 | −9 | −20 |
| Fusiform_R | 3.79E−05 | 30 | −2 | −30 |
| Cingulum_Mid_L | 1.26E−04 | −14 | −18 | 38 |
| Frontal_Mid_L | 1.62E−04 | −24 | 13 | 25 |
| Fusiform_R | 2.47E−04 | 30 | −2 | −34 |
| Insula_R | 2.52E−04 | 42 | −14 | −6 |
| Temporal_Inf_R | 9.54E−04 | 30 | 2 | −40 |

*Survived multiple comparisons FWE = 0.050
Total number of voxels which survived uncorrected p = 0.001 is 2136

Demonstration of the activity of the compounds of the present invention may be accomplished through in vitro, ex vivo and in vivo assays that are well known in the art. The effect of T3D-959 on the FDG-PET outcomes appears to be T3D-959 dose-dependent, with larger effects observed at larger doses. This observation comes from the sROI, and anatomical ROI analyses as well as the exploratory voxel-wise SPM analysis. The image displays below from the voxel-wise analysis shows a clear increase in the spatial extent of the regions of the yellow regions from 70 voxels to 2136 voxels as the dose increases from 10 mg to 90 mg. The yellow regions are made up of voxels with statistically significant differences from baseline to end of treatment (ROSDs).

### DETAILED DESCRIPTION OF THE DRAWINGS

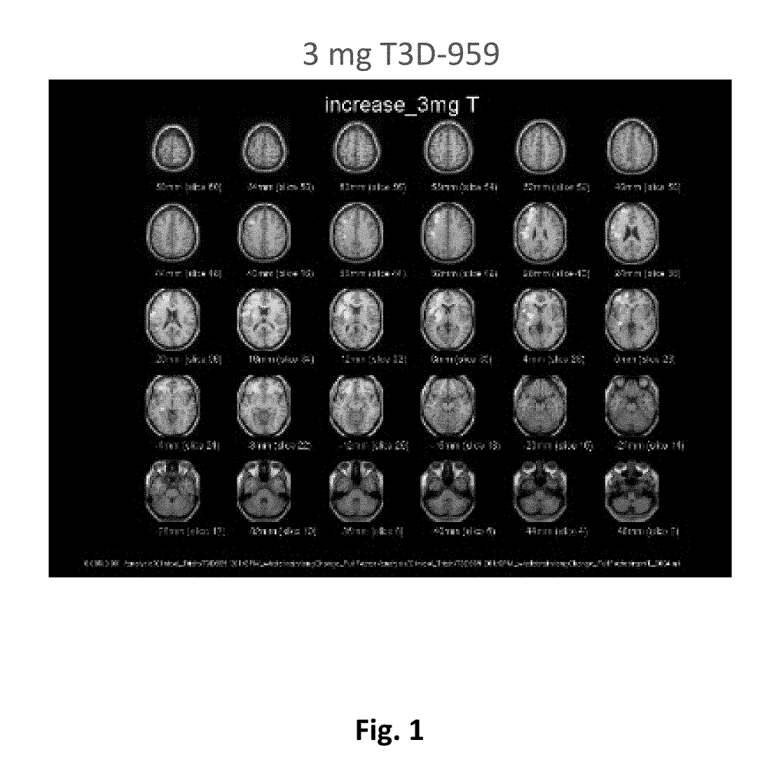

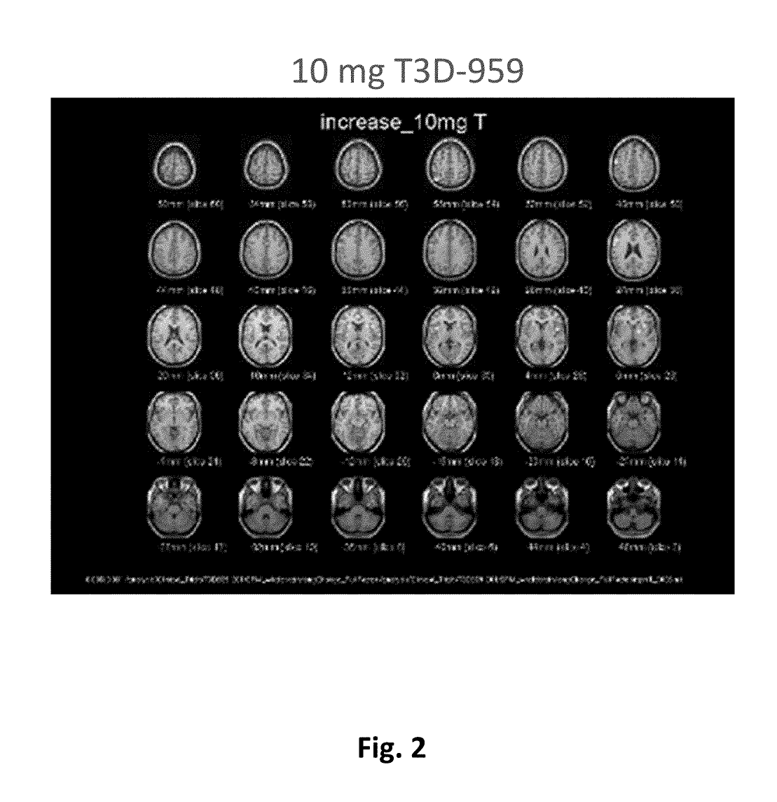

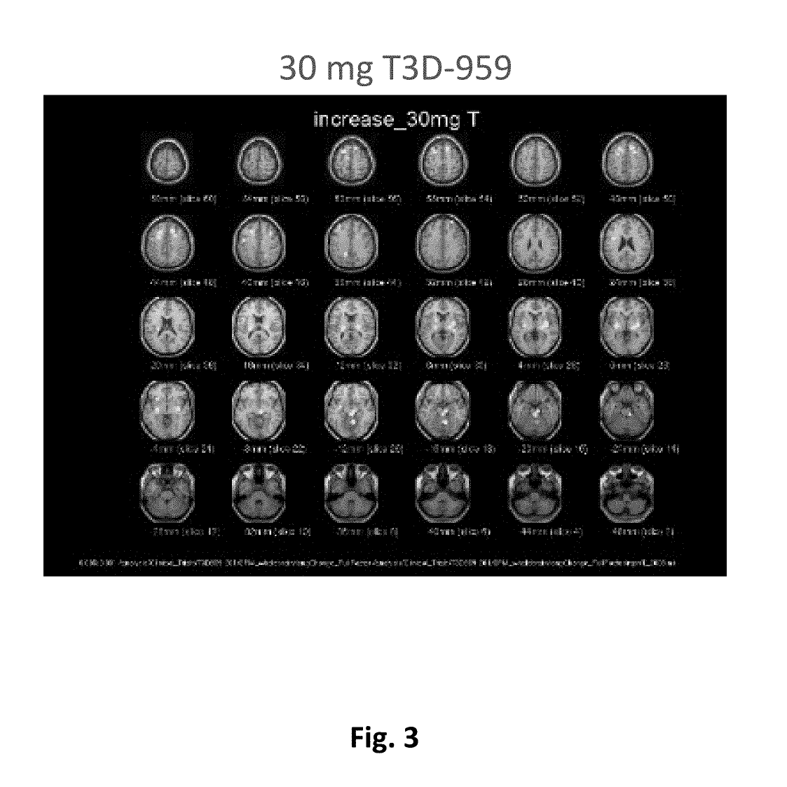

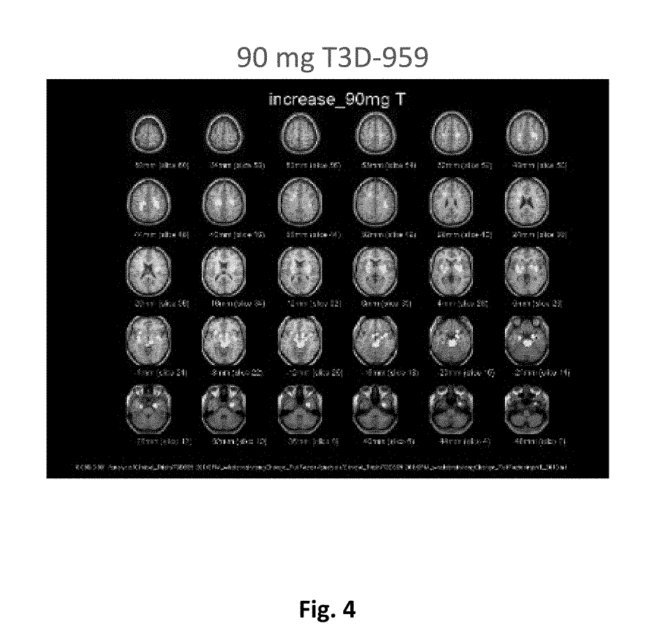

ROSDs with Positive Δ R CMRgI (EOT-BL) Relative to Whole Brain (p<0.005) for with Increasing Doses of T3D-959 are shown at 3 mg, 10 mg, 30 mg, and 40 mg doses in FIGS. 1-4 respectively.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

1. A method of treating a subject having a neurodegenerative disease comprising administering an indane acetic acid, dual Peroxisome Proliferator-Activated Receptor (PPAR) delta and gamma agonist, which penetrates the blood brain barrier (BBB) and achieves pharmacologically useful concentrations in the brain, wherein the neurodegenerative disease is selected from the group comprising:
j) Alzheimer's Disease (AD);
k) Huntington's Disease (HD);
l) Parkinson's Disease (PD);
m) Amyotrophic Lateral Sclerosis (ALS);
n) Frontal Temporal Dementia (FTD);
o) Corticobasal Degeneration (CBD);
p) Progressive Supranuclear Palsey (PSP);
q) Dementia with Lewy Bodies (DLB);
r) Multiple Sclerosis (MS);
and wherein, the indane acetic acid, comprises a compound of Formula I, or a pharmaceutically acceptable salt, ester prodrug, stereoisomer, enantiomer, racemate or a combination thereof wherein Formula I is: 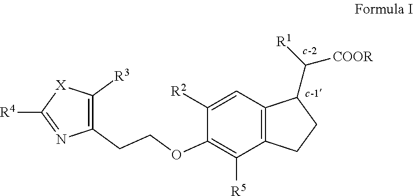
wherein
R is H, Na⁺, Li⁺, Ca⁺, K⁺, N⁺(C1-C6)₄, or C1-C6 alkyl;
R¹ is H, COOR, C₃-C₈ cycloalkyl, or C₁-C₆ alkyl, C₂-C₆ alkenyl, or C₁-C₆ alkoxy, each of which may be unsubstituted or substituted with fluoro, methylenedioxyphenyl, or phenyl which may be unsubstituted or substituted with R⁶, “c-2” is defined as the second carbon of the acetic acid portion of Formula I, and “c-1′” is the first carbon of the indane group of Formula I;
R² is H, halo, or C₁-C₆ alkyl which may be unsubstituted or substituted with C₁-C₆ alkoxy, oxo, fluoro,
or R² is phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, or morpholinyl, each of which may be unsubstituted or substituted with R⁶;
R³ is H, C₁-C₆ alkyl, or phenyl, which may be unsubstituted or substituted with R⁶;
X is O or S;
R⁴ is phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl, each of which may be unsubstituted or singularly or multiply substituted with R⁶, or with phenyl, furyl, thienyl, pyrrolyl, oxazolyl, thiazolyl, imidazolyl, pyrazolyl, isoxazolyl, isothiazolyl, triazolyl, oxadiazolyl, thiadiazolyl, tetrazolyl, pyridyl, pyrrolidinyl, piperidinyl, tetrahydropyranyl, tetrahydrothiopyranyl, piperazinyl, morpholinyl, benzodioxolyl, dihydrobenzofuranyl, indolyl, pyrimidinyl or phenoxy, each of which may be unsubstituted or singularly or multiply substituted with R⁶;
or R⁴ is C₁-C₆ alkyl or C₃-C₈ cycloalkyl, either of which may be unsubstituted or substituted with fluoro, oxo, or C₁-C₆ alkoxy which may be unsubstituted or substituted with C₁-C₆ alkoxy, or phenyl optionally substituted with R⁶, each of which may be substituted with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benzothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl, each of which may be unsubstituted or further substituted with R⁶, or any C₁-C₆ alkyl may also be substituted with C₃-C₈ cycloalkyl or with phenoxy which may be unsubstituted or substituted with R⁶ or with phenyl, naphthyl, furyl, thienyl, pyrrolyl, tetrahydrofuryl, pyrrolidinyl, pyrrolinyl, tetrahydrothienyl, oxazolyl, thiazolyl, imidazolyl, pyrazolyl, isoxazolyl, isothiazolyl, triazolyl, oxadiazolyl, thiadiazolyl, tetrazolyl, pyridyl, piperidinyl, tetrahydropyranyl, tetrahydrothiopyranyl, pyrimidinyl, pyrazinyl, pyridazinyl, piperazinyl, morpholinyl, benzofuryl, dihydrobenzofuryl, benzothienyl, dihydrobenzothienyl, indolyl, indolinyl, indazolyl, benzoxazolyl, benxothiazolyl, benzimidazolyl, benzisoxazolyl, benzisothiazolyl, benzodioxolyl, quinolyl, isoquinolyl, quinazolinyl, quinoxazolinyl, dihydrobenzopyranyl, dihydrobenzothiopyranyl, or 1,4-benzodioxanyl, each of which may be unsubstituted or substituted with R⁶, or
R⁵ is H, halo or C₁-C₆ alkyl optionally substituted with oxo; and
R⁶ is halo, CF₃, C₁-C₆ alkyl optionally substituted with oxo or hydroxy, or C₁-C₆ alkoxy optionally substituted with fluoro; and wherein R³ may be attached to the heterocyclic moiety of the compound of Formula I at either the 4 or 5 position, and, accordingly, the remaining portion of the molecule will be attached at the remaining available carbon atom.

2. The method of treatment according to claim 1, wherein the dual PPAR delta and gamma agonist, which penetrates the blood brain barrier (BBB), and achieves pharmacologically useful concentrations in the brain, is of Formula I, or is a pharmaceutically acceptable salt, ester prodrug, stereoisomer, enantiomer, racemate or a combination thereof wherein Formula I is: 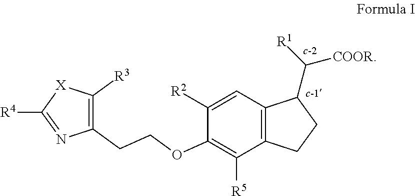
wherein
R is H, Na⁺, Li⁺, Ca⁺, K⁺, N⁺(C1-C6)₄, or C1-C6 alkyl;
R¹ is H, “c-2” is defined as the second carbon of the acetic acid portion of Formula I, and “c-1′” is the first carbon of the indane group of Formula I;
R² is H, halo, or C₁-C₆ alkyl which may be unsubstituted or substituted with C₁-C₆ alkoxy, oxo, fluoro;
R³ is H, C₁-C₆ alkyl, or phenyl, which may be unsubstituted or substituted with R⁶;
X is O or S;
R⁴ is phenyl, which may be unsubstituted or singularly or multiply substituted with R⁶;
R⁵ is H, halo or C₁-C₆ alkyl optionally substituted with C₁-C₆ alkoxy, oxo, fluoro;
R⁶ is halo, CF₃, C₁-C₆ alkyl optionally substituted with oxo or hydroxy, or
C₁-C₆ alkoxy optionally substituted with fluoro; wherein R³ may be attached to the heterocyclic moiety of the compound of Formula I at either the 4 or 5 position and, accordingly, the remaining portion of the molecule will be attached at the remaining available carbon atom.

3. The method of treatment according to claim 2 wherein the indane acetic acid which penetrates the blood brain barrier has a rat brain to plasma ratio of greater than 10% 12 hours after oral dosing.

4. The method of treatment according to claim 3 wherein the indane acetic acid is used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS):

5. The method of treatment according to claim 1 wherein:
R is H, Na⁺, Li⁺, Ca⁺, K⁺, N⁺(C1-C6)₄, or C1-C6 alkyl;
R¹ is H;
R² is H, halo;
R³ is H, C₁-C₆ alkyl;
X is O or S;
R⁴ is phenyl, which may be singularly or multiply substituted with R⁶;
R⁵ is H, halo;
R⁶ is halo, CF₃, C₁-C₆ alkyl or C₁-C₆ alkoxy; and
c-1′ has the S stereochemistry.

6. The method of treatment according to claim 5 wherein the indane acetic acid which penetrates the blood brain barrier has a rat brain to plasma ratio of greater than 10% 12 hours after oral dosing.

7. The method of treatment according to claim 6 wherein the indane acetic acid is used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS).

8. The method of treatment according to claim 1 wherein:
R is H, Na⁺, Li⁺, Ca⁺, K⁺, N⁺(C1-C6)₄, or C1-C6 alkyl;
R¹ is H;
R² is H, halo;
R³ is C₁-C₆ alkyl;
X is O;
R⁴ is phenyl, which may be singularly or multiply substituted with R⁶;
R⁵ is H, halo;
R⁶ is halo, CF₃, C₁-C₆ alkyl or C₁-C₆ alkoxy; and c-1′ has the S stereochemistry.

9. The method of treatment according to claim 8 wherein the indane acetic acid which penetrates the blood brain barrier has a rat brain to plasma ratio of greater than 10% 12 hours after oral dosing.

10. The method of treatment according to claim 9 wherein the indane acetic acid is used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS).

11. The method of treatment according to claim 1 wherein:
R is H, Na⁺, Li⁺, Ca⁺, K⁺, N⁺(C1-C6)₄, or C1-C6 alkyl;
R¹ is H,
R² is F,
R⁵ is F,
R³ is C₁-C₆ alkyl,
X is O or S, and
R⁴ is a phenyl, singularly or multiply substituted with R⁶, wherein R⁶ is halo, CF₃, C₁-C₆ alkoxyl or C₁-C₆ alkyl, and the stereochemistry at c-1′ is defined as S.

12. The method of treatment according to claim 11 wherein the indane acetic acid which penetrates the blood brain barrier has a rat brain to plasma ratio of greater than 10% 12 hours after oral dosing.

13. The method of treatment according to claim 12 wherein the indane acetic acid is used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS).

14. The method of treatment according to claim 1 wherein:
R is H or Na,
R¹ is H,
R² is H,
R⁵ is H,
R³ is C₁-C₆ alkyl,
X is O, and
R⁴ is a phenyl, singularly or multiply substituted with R⁶, wherein R⁶ is halo, CF₃, C₁-C₆ alkoxyl or C₁-C₆ alkyl, and the stereochemistry at C-1′ is defined as S.

15. The method of treatment according to claim 14 wherein the indane acetic acid which penetrates the blood brain barrier has a rat brain to plasma ratio of greater than 10% 12 hours after oral dosing.

16. The method of treatment according to claim 15 wherein the indane acetic acid is used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS).

17. The method of treatment according to claim 1 wherein:
R is H or Na,
R¹ is H,
R² is H,
R⁵ is H,
R³ is C₁-C₆ alkyl,
X is S, and
R⁴ is a phenyl, singularly or multiply substituted with R⁶,
wherein R⁶ is halo, CF₃, C₁-C₆ alkoxyl or C₁-C₆ alkyl, and
the stereochemistry at c-1′ is defined as S.

18. The method of treatment according to claim 17 wherein the indane acetic acid which penetrates the blood brain barrier has a rat brain to plasma ratio of greater than 10% 12 hours after oral dosing.

19. The method of treatment according to claim 18 wherein the indane acetic acid is used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS).

20. The method of treatment according to claim 1 wherein the PPAR dual delta and gamma agonist, which penetrates the blood brain barrier (BBB), is selected from the group consisting of the free acid, in the form of a pharmaceutically acceptable salt selected from the group consisting of a potassium, sodium, calcium, magnesium, lysine, choline or meglumine salt thereof, and has a structure selected from the group consisting of:

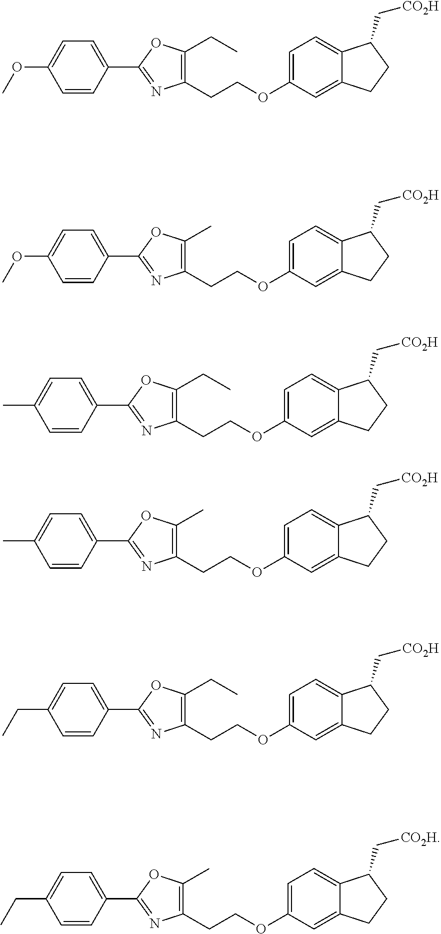 [SMILES: CCc1oc(-c2ccc(OC)cc2)nc1CCOc1ccc2c(c1)CC[C@H]2CC(=O)O ; COc1ccc(-c2nc(CCOc3ccc4c(c3)CC[C@H]4CC(=O)O)c(C)o2)cc1 ; CCc1oc(-c2ccc(C)cc2)nc1CCOc1ccc2c(c1)CC[C@H]2CC(=O)O ; Cc1ccc(-c2nc(CCOc3ccc4c(c3)CC[C@H]4CC(=O)O)c(C)o2)cc1 ; CCc1ccc(-c2nc(CCOc3ccc4c(c3)CC[C@H]4CC(=O)O)c(CC)o2)cc1 ; CCc1ccc(-c2nc(CCOc3ccc4c(c3)CC[C@H]4CC(=O)O)c(C)o2)cc1]

21. The method of treatment according to claim 20 wherein the indane acetic acid which penetrates the blood brain barrier has a rat brain to plasma ratio of greater than 20% 12 hours after oral dosing.

22. The method of treatment according to claim 21 wherein the indane acetic acid is used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS).

23. The method of treatment according to claim 22 wherein the indane acetic acid, which penetrates the blood brain barrier (BBB), is a pharmaceutically acceptable salt thereof, wherein the pharmaceutically acceptable salt is selected from the group consisting of ammonium salts, protonated basic amines, and quaternary amines.

24. The method of treatment according to claim 1 wherein the indane acetic acid, which penetrates the blood brain barrier (BBB), and is used to treat a neurodegenerative disease, wherein administration is selected from the group consisting of intravenously, orally, buccally, transdermally, rectally, nasally, optically, intrathecally and intra-cranially.

25. The method of treatment according to claim 2 which further comprises one or more additional therapeutic agents elected from the group consisting of:
a) a therapeutic agent used to treat Alzheimer's disease selected from the group consisting of Donepezil, Rivastigmine, or Memantine;
b) a therapeutic agent used to treat Parkinson's Disease (PD) selected from the group consisting of L DOPA (levodopa), or dopamine agonists;
c) a therapeutic agent used to treat Amyotrophic Lateral Sclerosis (ALS) selected from the group consisting of Edaravone (Radicut), or Riluzole;
d) a therapeutic agent used to treat Multiple Sclerosis (MS) selected from the group consisting of interferon beta, glatiramer acetate, mitoxantrone, natalizumab, fingolimod, teriflunomide, alemtuzumab, daclizumab, or ocrelizumab; and
e) a therapeutic agent used to treat Huntington's Disease (HD) is tetrabenazine.

26. The method of treatment according to claim 1 wherein the indane acetic acid, which penetrates the blood brain barrier (BBB), and is used to treat the listed neurodegenerative diseases is: ((1S)-5-{5-ethyl-2-(4-methoxyphenyl)-1, 3-oxazol-4-yl] ethoxy}-2, 3-dihydro-1H-inden-1-yl) acetic acid, sodium salt (CAS Registry number 1258076-66-2) the following structure:

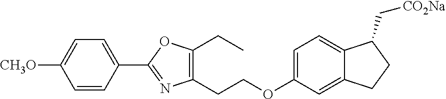

27. The method of treatment according to claim 26 wherein the indane acetic acid which penetrates the blood brain barrier has a rat brain to plasma ratio of greater than 20% 12 hours after oral dosing.

28. The method of treatment according to claim 27 wherein the indane acetic acid is used to treat a neurodegenerative disease selected from Huntington's Disease (HD); Parkinson's Disease (PD); Amyotrophic Lateral Sclerosis (ALS); Frontal Temporal Dementia (FTD); Corticobasal Degeneration (CBD); Progressive Supranuclear Palsey (PSP); Dementia with Lewy Bodies (DLB); or Multiple Sclerosis (MS).

29. The method of treatment according to claim 27 wherein the indane acetic acid, which penetrates the blood brain barrier (BBB), wherein administration is selected from the group consisting of intravenously, orally, buccally, transdermally, rectally, nasally, optically, intrathecally and intra-cranially.

30. The method of treatment according to claim 27 which further comprises one or more additional therapeutic agents selected from the group consisting of:
a) a therapeutic agent used to treat Alzheimer's disease selected from the group consisting of Donepezil, Rivastigmine, or Memantine;
b) a therapeutic agent used to treat Parkinson's Disease (PD) selected from the group consisting of L DOPA (levodopa), or dopamine agonists;
c) a therapeutic agent used to treat Amyotrophic Lateral Sclerosis (ALS) selected from the group consisting of Edaravone (Radicut), or Riluzole;
d) a therapeutic agent used to treat Multiple Sclerosis (MS) selected from the group consisting of interferon beta, glatiramer acetate, mitoxantrone, natalizumab, fingolimod, teriflunomide, alemtuzumab, daclizumab, or ocrelizumab; and
e) a therapeutic agent used to treat Huntington's Disease (HD) is tetrabenazine.
